(19) United States
(12) Patent Application Publication
(10) Pub. No.: US 2003/0144249 A1
(43) Pub. Date: Jul. 31, 2003

(54) Use of organophosphorous compounds for producing a medicament for treating infections

(76) Inventor: Hasaan Jomaa, Giessen (DE)

Correspondence Address: WILLIAM COLLARD, COLLARD & ROE, P.C., 1077 NORTHERN BOULEVARD, ROSLYN, NY, 11576, US

(21) Appl. No.: 10/239,040

(22) PCT Filed: Mar. 21, 2001
(86) PCT No.: PCT/EP01/03201

(30) Foreign Application Priority Data
Mar. 22, 2000 (DE) 100 14 127.7

(51) Int. Cl. A61K 31/675; A61K 31/66

(52) U.S. Cl. 514/79; 514/114

(57) ABSTRACT

The invention relates to the use of organophosphorus compounds of the general formula (I)

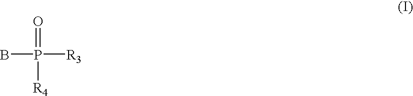

wherein B is selected from the group which consists of group

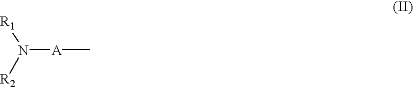

and group

R₁—N═A— (III)

and wherein A is selected from the group which consists of an alkyleneamine residue, an alkenyleneamine residue, a hydroxyalkyleneamine residue, an alkyleneimine residue, an alkenyleneimine residue and a hydroxyalkyleneimine residue, wherein the nitrogen atom is located in the chain which links the phosphorus atom with the nitrogen atom of the group

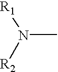

or group R₁—N═, for the therapeutic and prophylactic treatment of infections in humans and animals caused by viruses, bacteria, fungi and parasites and pharmaceutical preparations which contain these compounds as the active ingredient, and to the use thereof as a fungicide, bactericide and herbicide in plants.

The invention relates to the use of organophosphorus compounds and the salts, esters and amides thereof for the therapeutic and prophylactic treatment of infections in humans and animals which are caused by viruses, bacteria, fungi and parasites, to pharmaceutical agents containing said compounds and to the use thereof as a fungicide, bactericide and herbicide in plants. According to the invention, the organophosphorus compound comprise phosphinoyl derivatives, phosphinic acid derivatives and phosphonic acid derivatives.

In order to widen the range of options for treating humans and animals and for protecting plants, there is an urgent requirement to provide agents which are not only highly active but, unlike other pharmaceutical preparations or phytosanitary agents, also exhibit reduced side-effects or reduced environmental impact and thus constitute a reduced risk to human health.

The object of the present invention is accordingly to provide a substance which is universally usable in infections by viruses, bacteria, fungi and parasites in humans and animals and as a fungicide, bactericide and herbicide in plants and which meets the above-stated requirements.

This object is utterly surprisingly achieved by the group of substances defined in claim 1. This group of substances exhibits both an antiinfective action against viruses, bacteria, fungi and uni- and multicellular parasites and a fungicidal, bactericidal and herbicidal action in plants. According to the invention, unicellular parasites should be taken to mean solely protozoa as narrowly defined in parasitology. Thanks to a C—N—C spacer, the fosmidomycin derivatives, which are known as antibiotics, exhibit improved absorption by microorganisms, as a result of which more rapid elimination of the parasites may be achieved in the case of malaria. This has the advantage that more rapid elimination of the parasites ensures that clinical symptoms subside more rapidly.

The substances have already been described in the literature for other applications, such as for example N′-(diphenylphosphinoylmethyl)-N,N-diethylethane-1,2-diamine in Tetrahedron; 53; 30; 1997; 10313-10330, 1,2,3-triazol-1-ylmethylphosphonic acid in Heterocycles; 40; 2; 1995; 545-550 and 3-phosphonomethyloctahydrobenzoimidazol-1-ylmethyl)phosphonic acid in Phosphorus, Sulfur Silicon Relat. Elem.; 101; 1-4; 1995; 131-140.

The organophosphorus compounds according to the invention are of the general formula (I):

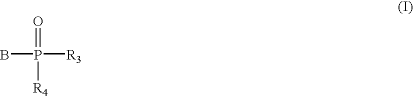

wherein R₃ and R₄ are identical or different and are selected from the group which consists of hydrogen, substituted and unsubstituted alkyl with up to 26 carbon atoms, substituted and unsubstituted hydroxyalkyl with up to 26 carbon atoms, substituted and unsubstituted aryl, substituted and unsubstituted acyl, substituted and unsubstituted aralkyl, substituted and unsubstituted alkenyl with up to 26 carbon atoms, substituted and unsubstituted alkynyl with up to 26 carbon atoms, substituted and unsubstituted cycloalkyl, substituted and unsubstituted heterocyclic residue, halogen, OX₃ or OX₄, wherein X₃ or X₄ may be identical or different and are selected from the group which consists of hydrogen, substituted and unsubstituted alkyl with up to 26 carbon atoms, substituted and unsubstituted hydroxyalkyl with up to 26 carbon atoms, substituted and unsubstituted aryl, substituted and unsubstituted aralkyl, substituted and unsubstituted alkenyl with up to 26 carbon atoms, substituted and unsubstituted alknyl with up to 26 carbon atoms, substituted and unsubstituted cycloalkyl, substituted and unsubstituted heterocyclic residue, a silyl, a cation of an organic and inorganic base, in particular a metal of main groups I, II or III of the periodic system, ammonium, substituted ammonium and ammonium compounds which are derived from ethylenediamine or amino acids, and B is selected from the group which consists of group (II)

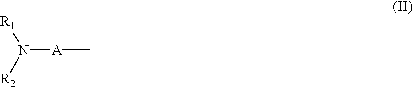

and group (III)

R₁—N═A— (III)

wherein A is selected from the group which consists of an alkyleneamine residue, an alkenyleneamine residue, a hydroxyalkyleneamine residue, an alkyleneimine residue, an alkenyleneimine residue and a hydroxyalkyleneimine residue, wherein the nitrogen atom is located in the chain which links the phosphorus atom with the nitrogen atom of the group

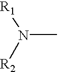

or the group R₁—N═, and in which R₁ and R₂ in group (II) are identical or different and R₁ and R₂ for group (II) and R₁ for group (III) are selected from the group which consists of hydrogen, substituted and unsubstituted alkyl, substituted and unsubstituted hydroxyalkyl, substituted and unsubstituted alkenyl, substituted and unsubstituted alkynyl, substituted and unsubstituted aryl, substituted and unsubstituted acyl, substituted and unsubstituted cycloalkyl, substituted and unsubstituted aralkyl, substituted and unsubstituted heterocyclic residue, halogen, OX₁ and OX₂, wherein X₁ and X₂ may be identical or different and are selected from the group which consists of hydrogen, substituted and unsubstituted alkyl, substituted and unsubstituted hydroxyalkyl, substituted and unsubstituted alkenyl, substituted and unsubstituted alkynyl, substituted and unsubstituted aryl, substituted and unsubstituted acyl, substituted and unsubstituted cycloalkyl, substituted and unsubstituted aralkyl, substituted and unsubstituted heterocyclic residue, and the pharmaceutically acceptable salts, esters and amides thereof and salts of the esters.

Preferably, A is an amino group in which the nitrogen atom is not in terminal position. Preferably, A links the nitrogen and the phosphorus atom with three atoms (without substituents).

Examples of substituents on the carbon atoms and on the nitrogen atom are hydroxy residues, alkyl residues, aryl residues, nitrogen heterocycles, acid residues, amines, cycloalkyl residues, sulfone residues, acyl residues and acylanilidine residues. The carbon atoms may here also comprise two substituents.

In particular, preferred compounds are those of the formula (IV)

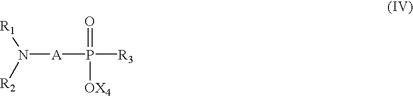

wherein

R₁, R₂, R₃ and X₄ are defined as for formula (I), and A is selected from the group which consists of C—N—C, C═N—C, C—N═C, wherein the carbon atoms may be substituted with a hydroxy or alkyl group with up to 7 carbon atoms.

Particularly preferably, R₁ is a hydroxy group, R₂ is selected from the group which consists of acetyl and formyl, R₃ is selected from the group which consists of hydrogen, methyl, ethyl, hexadecanyl, octadecanyl, alkoxyaryl and OX₃, and X₃ and X₄ are selected from the group which consists of hydrogen, sodium, potassium, methyl, ethyl, hexadecanyl, octadecanyl and alkoxyaryl and, when both are present, may be identical or different.

Further preferred compounds are those of the formula (V)

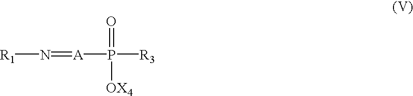

wherein

R₁, R₃ and X₄ are defined as for formula (I), and A is selected from the group which consists of C—N—C, C═N—C, C—N═C, wherein the carbon atoms may be substituted with a hydroxy or alkyl group with up to 7 carbon atoms.

Particularly preferably, R₁ is selected from the group which consists of acetyl and formyl and R₃ is selected from the group which consists of hydrogen, methyl, ethyl, hexadecanyl, octadecanyl, alkoxyaryl and OX₃, and X₃ and X₄ are selected from the group which consists of hydrogen, sodium, potassium, methyl, ethyl, hexadecanyl, octadecanyl and alkoxyaryl, and, when both are present, may be identical or different.

Examples of compounds which have proved advantageous are stated below. The compounds numbered from 1 to 8 have proved particularly suitable:

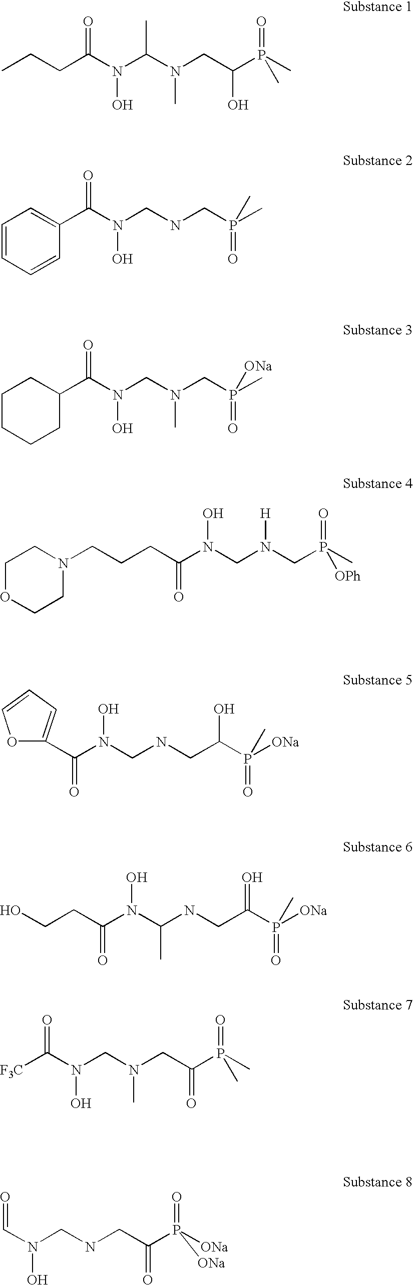

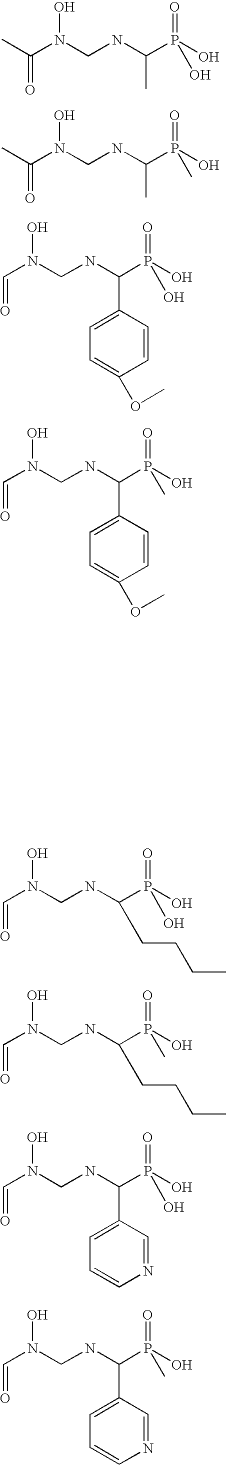

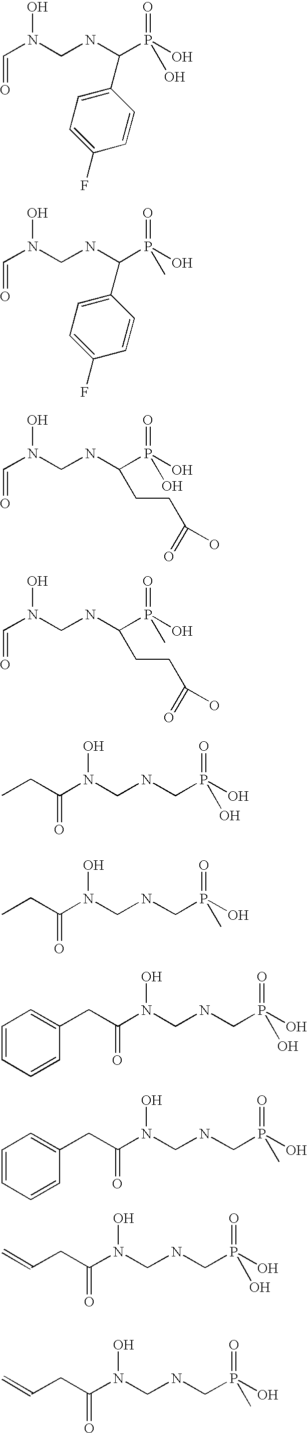

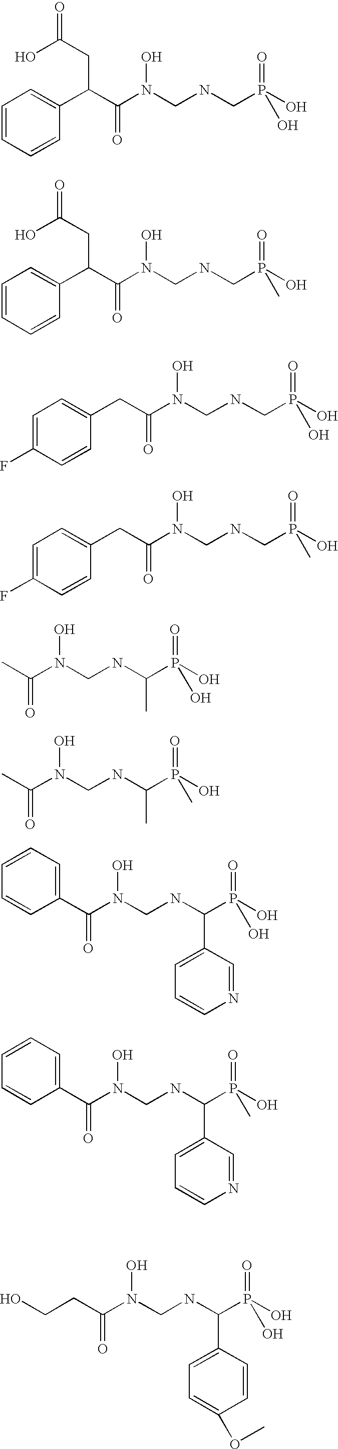

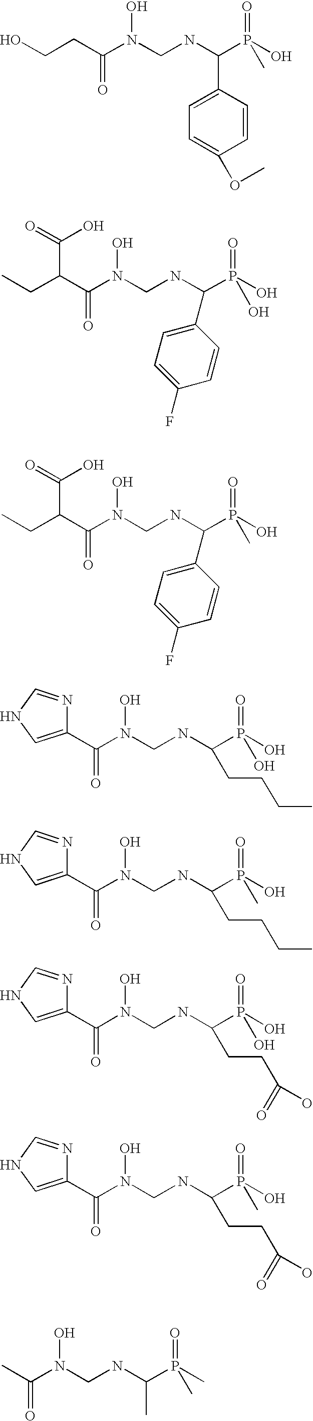

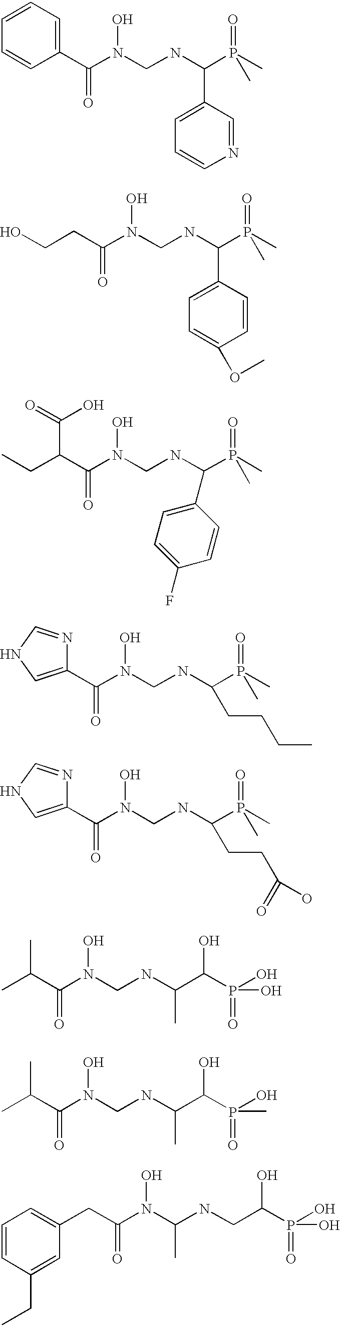

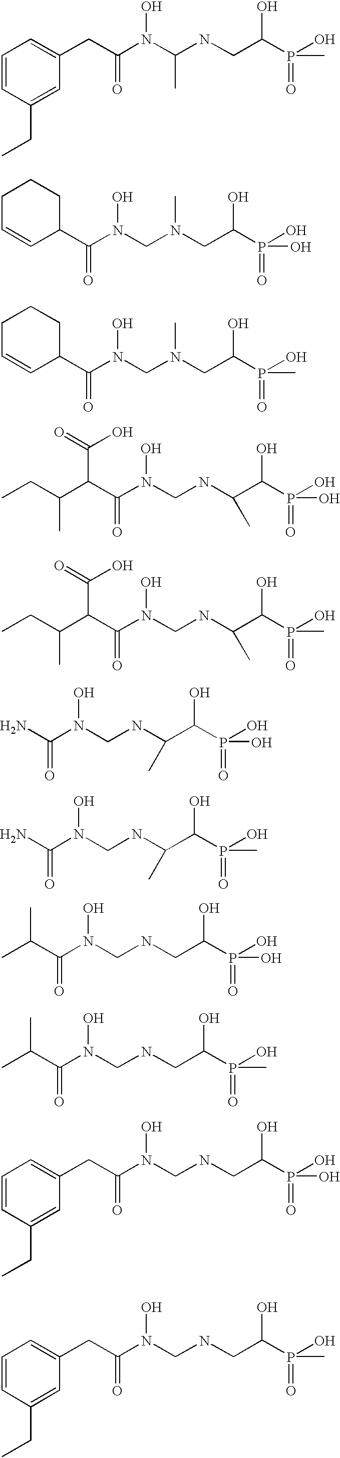

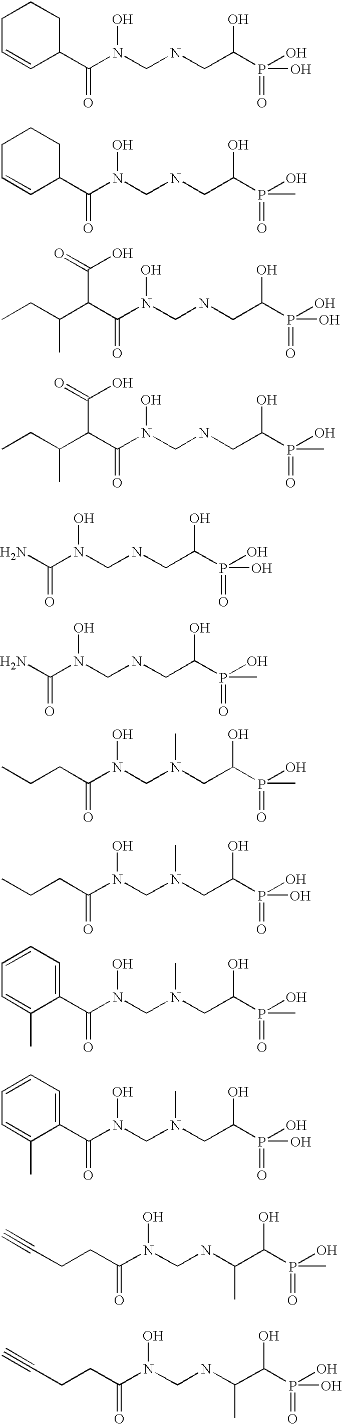

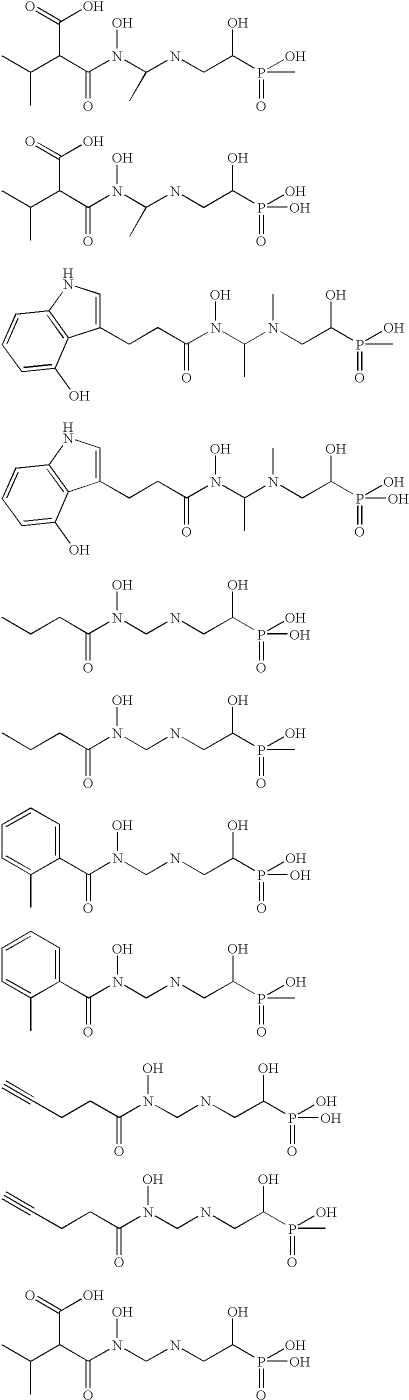

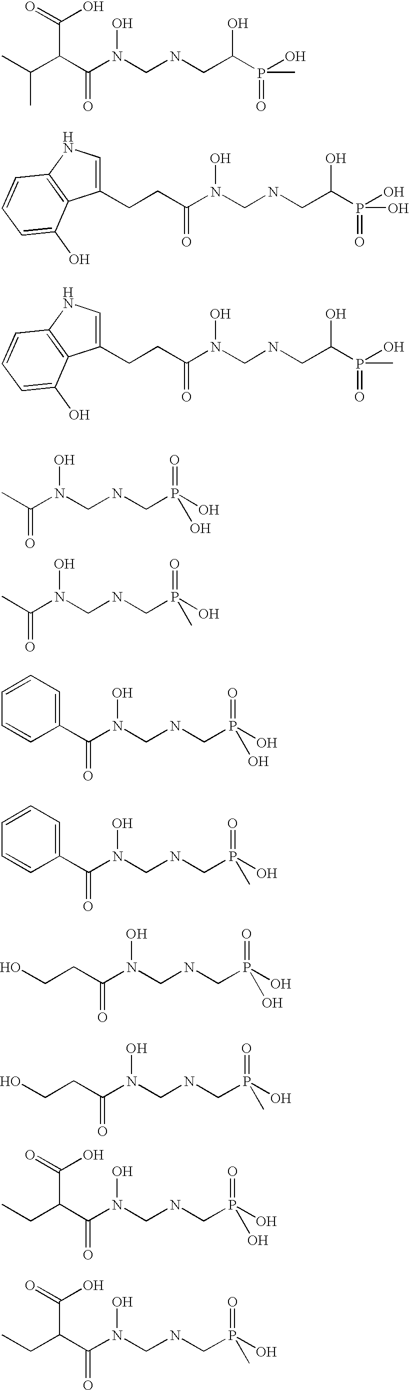

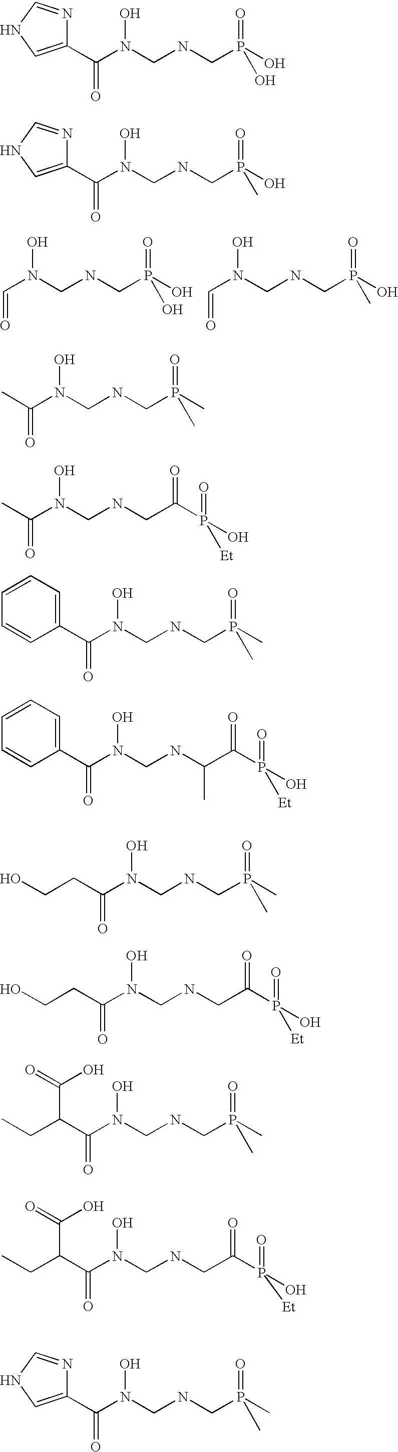

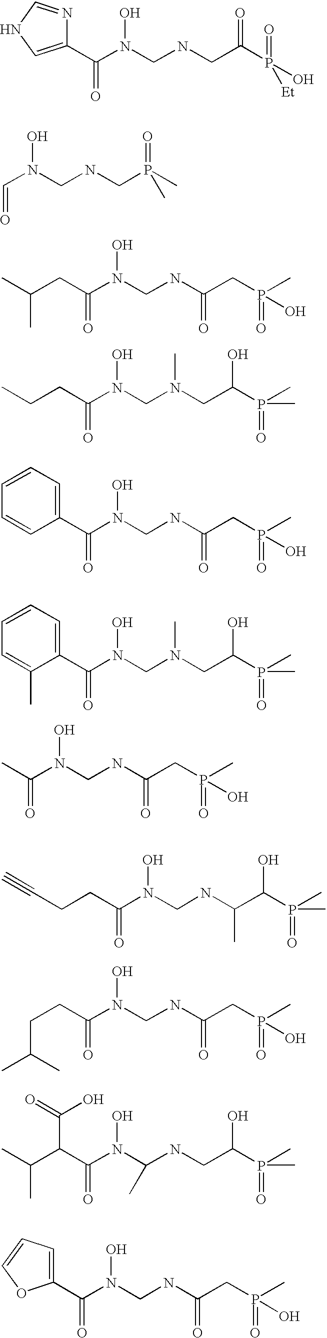

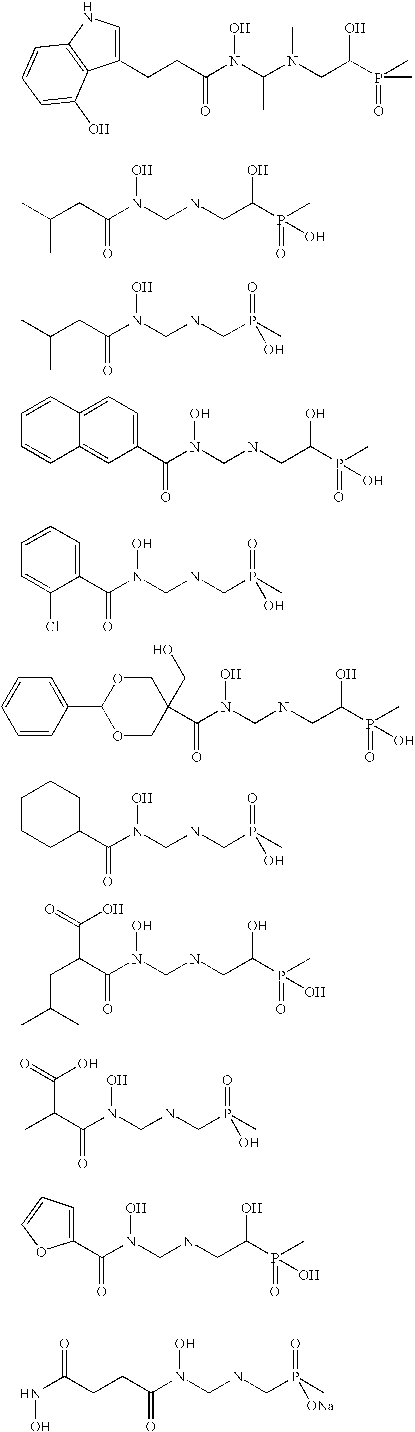

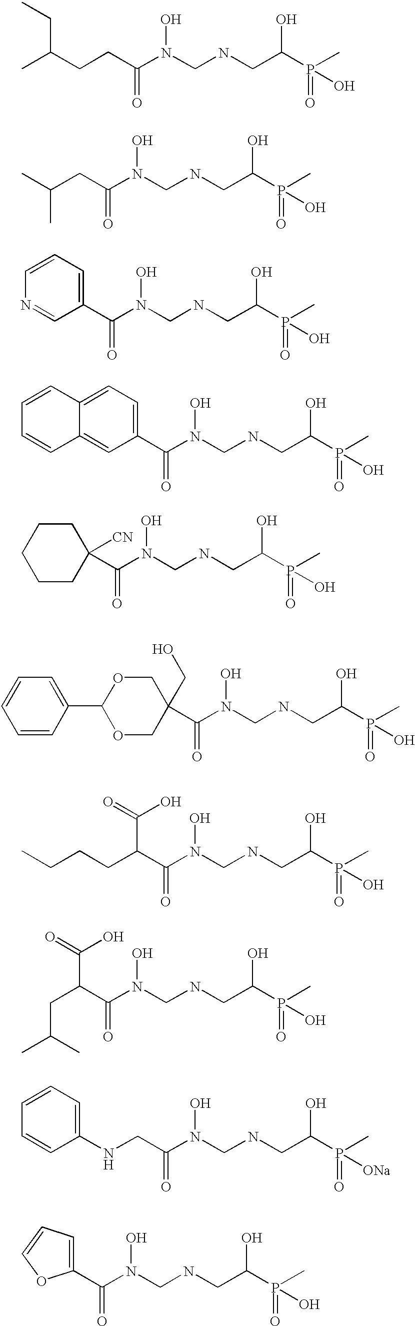

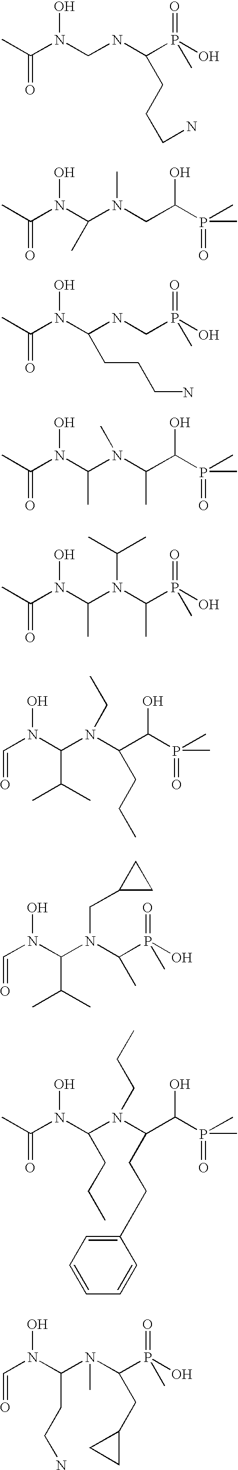

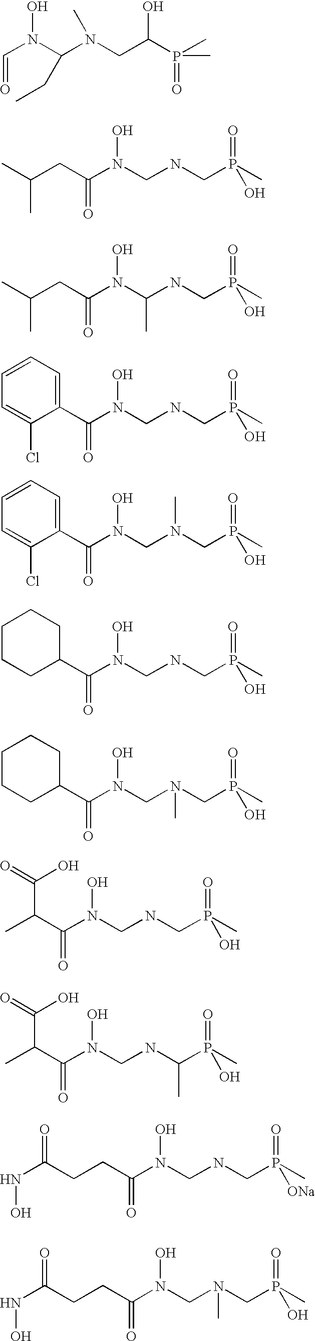

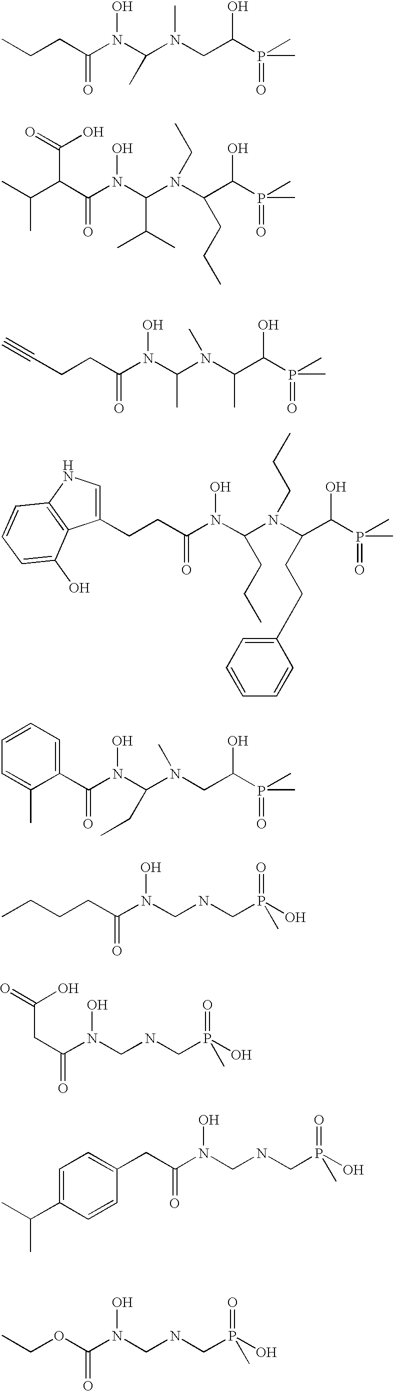

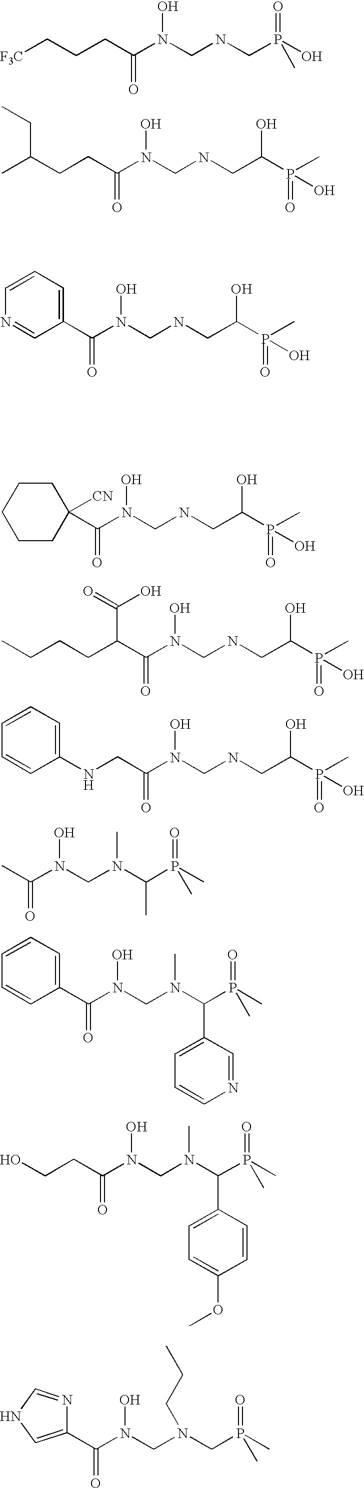

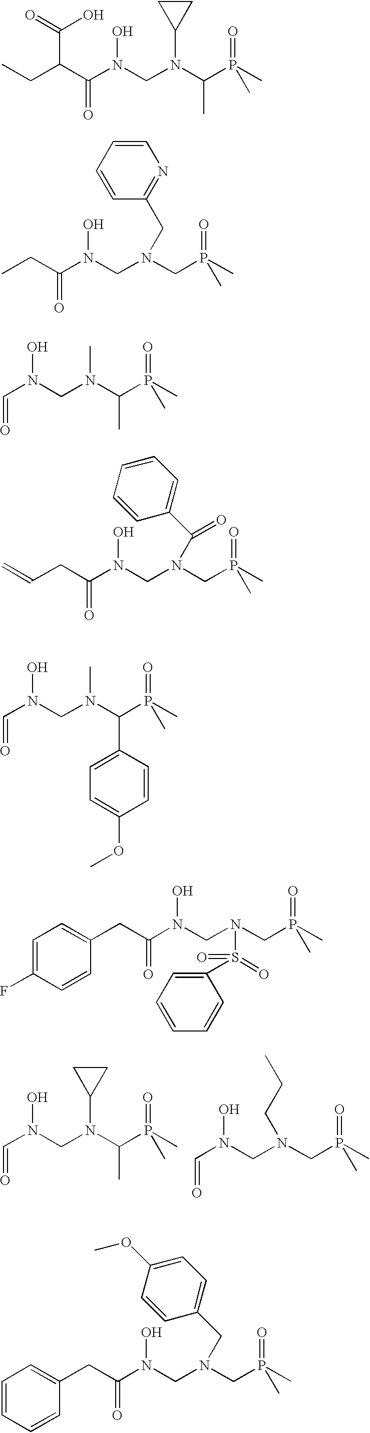

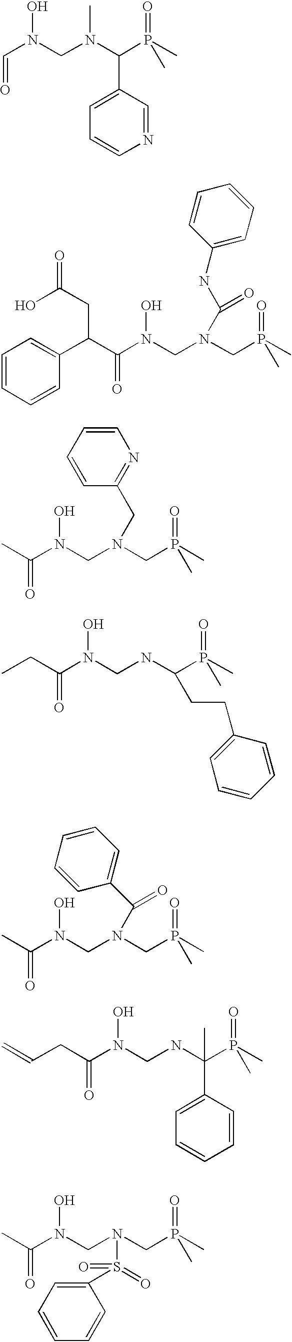

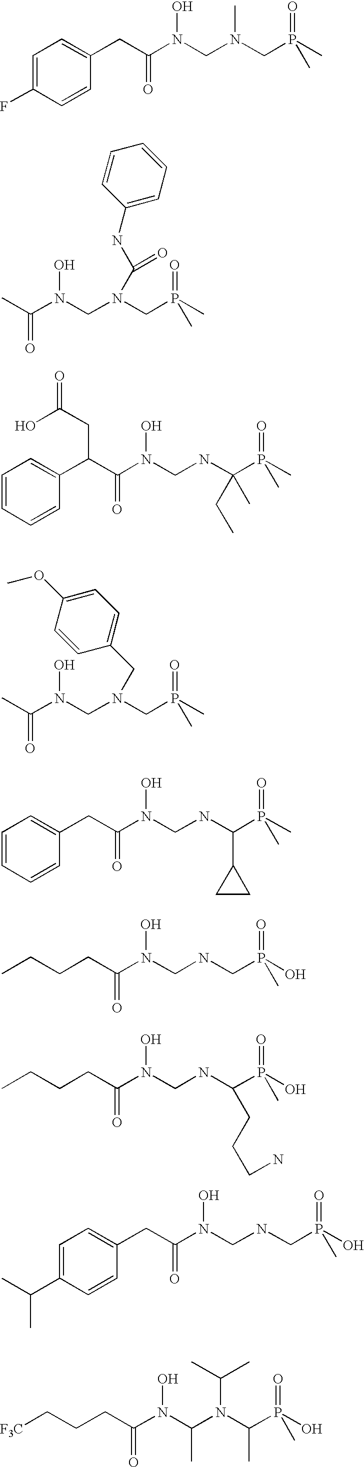

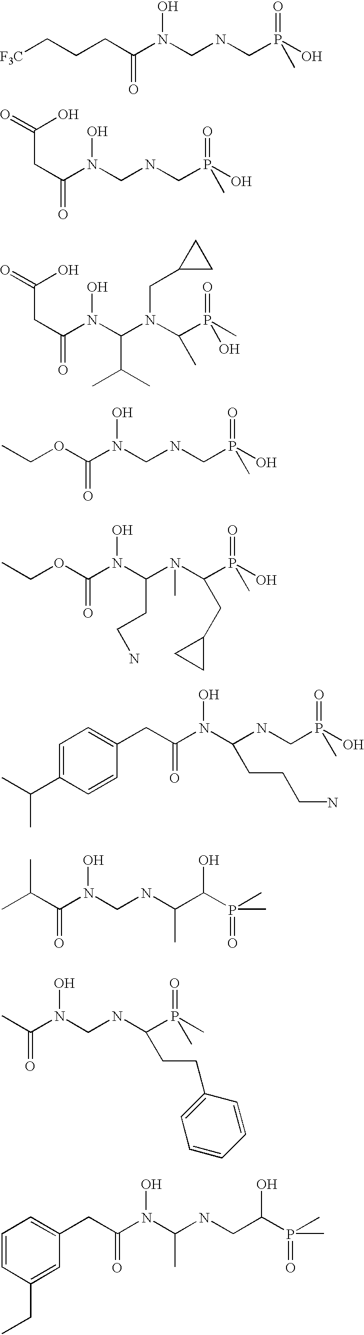

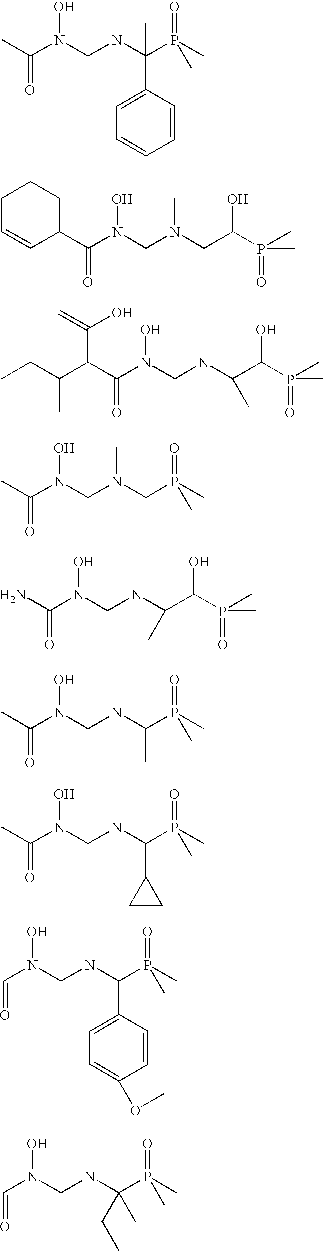

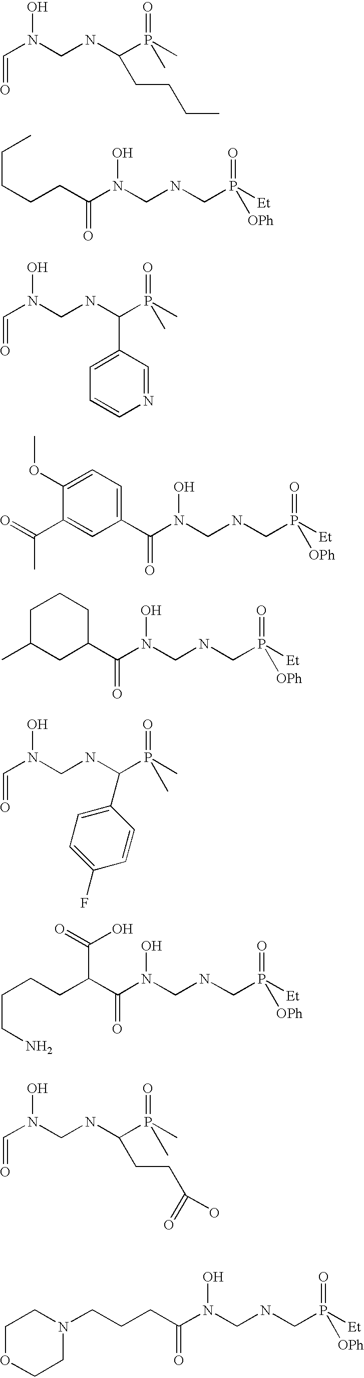

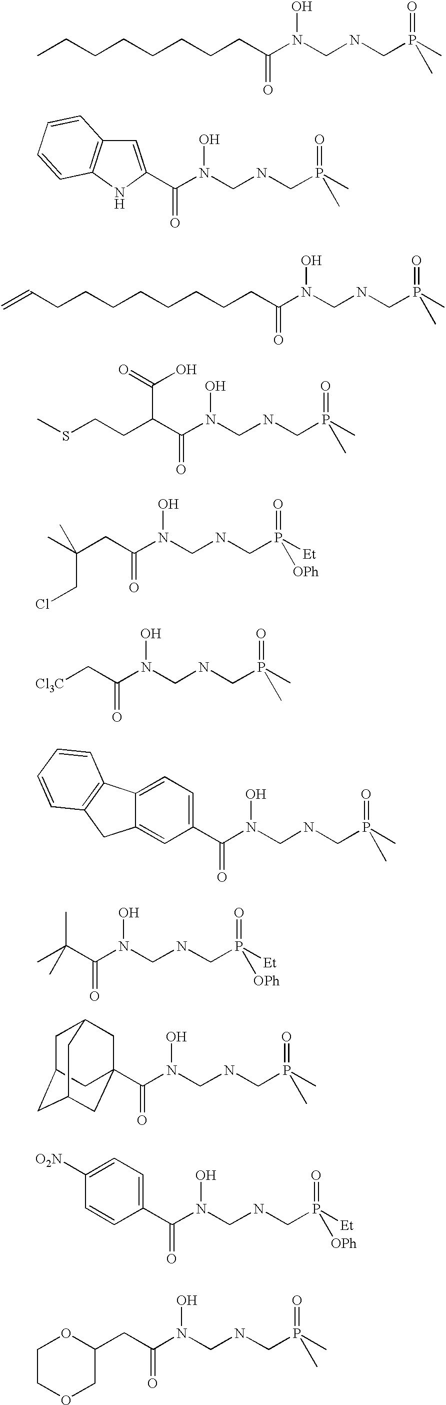

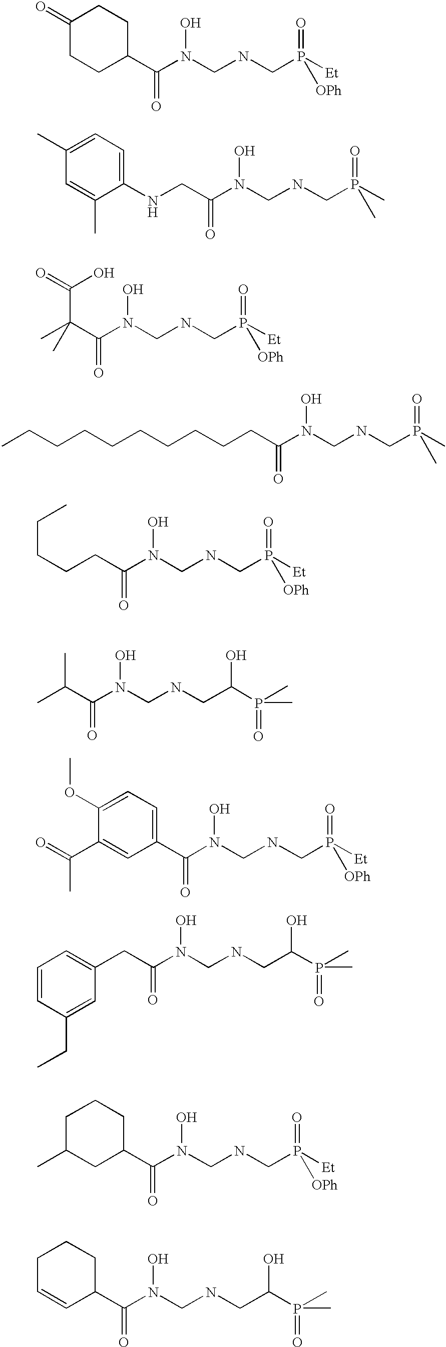

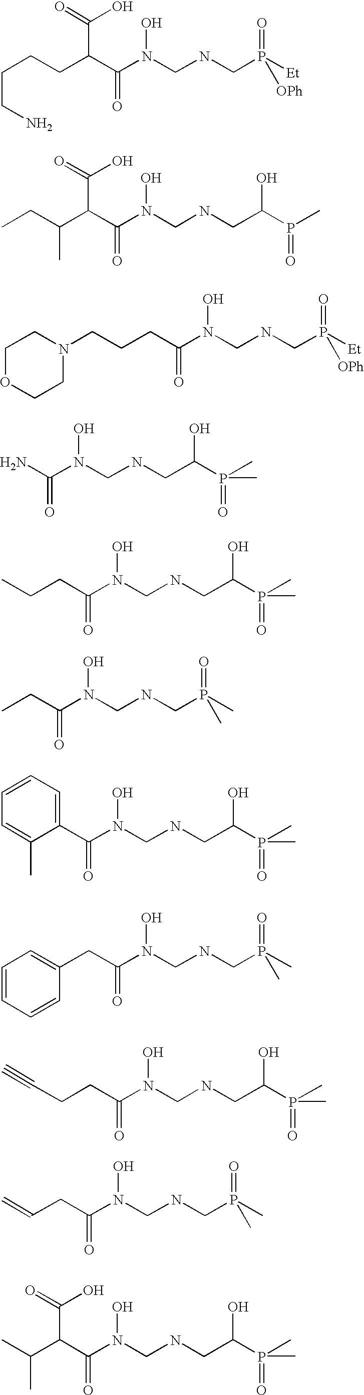

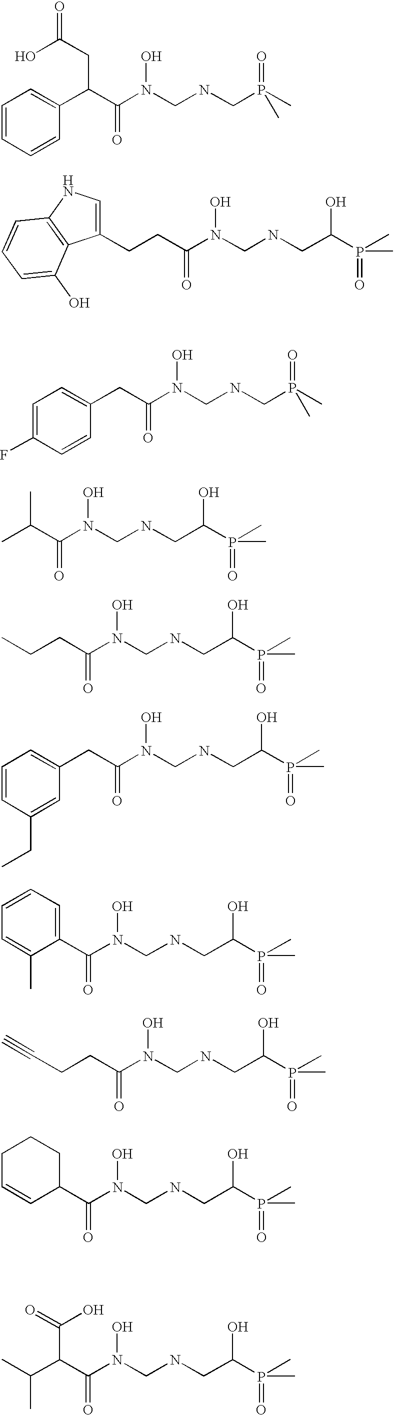

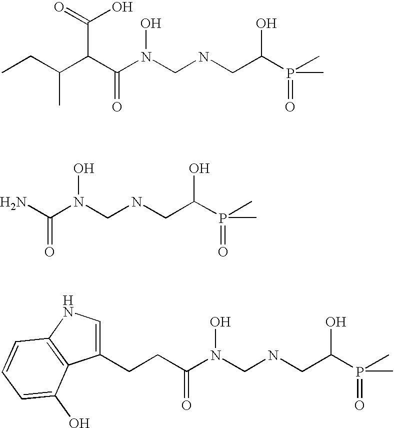

Special features of the above definitions and suitable examples thereof are given below:

“Acyl” is a substituent which originates from an acid, such as from an organic carboxylic acid, carbonic acid, carbamic acid or the thio acid or imidic acid corresponding to the above individual acids, or from an organic sulfonic acid, wherein these acids in each case comprise aliphatic, aromatic and/or heterocyclic groups in the molecule together with carbamoyl or carbamimidoyl.

Suitable examples of these acyl groups are given below.

Aliphatic acyl groups are defined as acyl residues originating from an aliphatic acid and include the following:

alkanoyl (for example formyl, acetyl, propionyl, butyryl, isobutyryl, valeryl, isovaleryl, pivloyl etc.);

alkenoyl (for example acryloyl, methacryloyl, crotonoyl etc.);

alkylthioalkanoyl (for example methylthioacetyl, ethylthioacetyl etc.)

alkanesulfonyl (for example mesyl, ethanesulfonyl, propanesulfonyl etc.);

alkoxycarbonyl (for example methoxycarbonyl, ethoxycarbonyl, propoxycarbonyl,

isoprpoxycarbonyl, butoxycarbonyl, isobutoxycarbonyl etc.);

alkylcarbamoyl (for example methylcarbamoyl etc.);

(N-alkyl)thiocarbamoyl (for example (N-methyl)thiocarbamoyl etc.);

alkylcarbamimidoyl (for example. methylcarbamimidoyl etc.);

oxalo;

alkoxalyl (for example methoxalyl, ethoxalyl, propoxalyl etc.).

In the above examples of aliphatic acyl groups, the aliphatic hydrocarbon moiety, in particular the alkyl group or alkane residue, may optionally have one or more suitable substituents, such as amino, halogen (for example fluorine, chlorine, bromine etc.), hydroxy, hydroxyimino, carboxy, alkoxy (for example methoxy, ethoxy, propoxy etc.), alkoxycarbonyl, acylamino (for example benzyloxycarbonylamino etc.), acyloxy (for example acetoxy, benzoyloxy etc.) and the like; preferred aliphatic acyl residues with such substituents which may be mentioned are, for example, alkanoyls substituted with amino, carboxy, amino and carboxy, halogen, acylamino or the like.

Aromatic acyl residues are defined as those acyl residues which originate from an acid with a substituted or unsubstituted aryl group, wherein the aryl group may comprise phenyl, toluyl, xylyl, naphthyl and the like; suitable examples are stated below:

aroyl (for example benzoyl, toluoyl, xyloyl, naphthoyl, phthaloyl etc.);

alkanoyl (for example phenylacetyl etc.);

aralkenoyl (for example cinnamoyl etc.);

aryloxyalkanoyl (for example phenoxyacetyl etc.);

arylthioalkanoyl (for example phenylthioacetyl etc.);

arylaminoalkanoyl (for example N-phenylglycyl, etc.);

arenesulfonyl (for example benzenesulfonyl, tosyl or toluenesulfonyl, naphthalenesulfonyl etc.);

aryloxycarbonyl (for example phenoxycarbonyl, naphthylbutoxycarbonyl etc.);

aralkoxycarbonyl (for example benzyloxycarbonyl etc.);

alkylcarbamoyl (for example phenylcarbamoyl, naphthylcarbamoyl etc.);

arylglyoxyloyl (for example phenylglyoxyloyl etc.).

In the above-stated examples of aromatic acyl residues, the aromatic hydrocarbon moiety (in particular the aryl residue) and/or the aliphatic hydrocarbon moiety (in particular the alkane residue) may optionally have one or more suitable substituents, such as those which have already been stated as suitable substituents for the alkyl group or the alkane residue. Examples of preferred aromatic acyl residues with specific substituents which may in particular be mentioned are aroyl substituted with halogen and hydroxy or with halogen and aralkanoyl substituted with hydroxy, hydroxyimino, dihaloalkanoyloxyimino, together with arylthiocarbamoyl (for example phenylthiocarbamoyl etc.); arylcarbamimidoyl (for example phenylcarbamimidoyl etc.).

A heterocyclic acyl residue is taken to mean an acyl residue which originates from an acid with a heterocyclic group; such residues include:

heterocyclic carbonyl, in which the heterocyclic residue is an aromatic or aliphatic 5- to 6-membered heterocycle with at least one heteroatom from the group nitrogen, oxygen and sulfur (for example thiophenyl, furoyl, pyrrolecarbonyl, nicotinyl etc.);

heterocycle-alkanoyl, in which the heterocyclic residue is 5- to 6-membered and comprises at least one heteroatom from the group nitrogen, oxygen and sulfur (for example thiophenylacetyl, furylacetyl, imidazolylpropionyl, tetrazolylacetyl, 2-(2-amino-4-thiazolyl)-2-methoxyiminoacetyl etc.) and the like.

In the above examples of heterocyclic acyl residues, the heterocycle and/or the aliphatic hydrocarbon moiety may optionally comprise one or more suitable substituents, such as the same as were stated to be suitable for alkyl and alkane groups.

“Alkyl” is a linear or branched alkyl residue with up to 9 carbon atoms, provided that it is not otherwise defined, such as methyl, ethyl, propyl, isopropyl, butyl, isobutyl, tert.-butyl, pentyl, hexyl and the like.

“Hydroxyalkyl” is a linear or branched alkyl residue with up to 9 carbon atoms, provided that it is not otherwise defined, which comprises at least one hydroxyl group, preferably one or two hydroxyl groups.

“Alkenyl” includes linear or branched alkenyl groups with up to 9 carbon atoms, provided that it is not otherwise defined, such as for example vinyl, propenyl (for example 1 -propenyl, 2-propenyl), 1-methylpropenyl, 2-methylpropenyl, butenyl, 2-ethylpropenyl, pentenyl, hexenyl.

“Alkynyl” includes linear or branched alkynyl groups with up to 9 carbon atoms, provided that it is not otherwise defined.

Cycloalkyl preferably denotes an optionally substituted C3-C7 cycloalkyl; possible substituents are inter alia alkyl, alkenyl, alkynyl, alkoxy (for example methoxy, ethoxy etc.), halogen (for example fluorine, chlorine, bromine etc.), nitro and the like.

Aryl is an aromatic hydrocarbon residue, such as phenyl, naphthyl etc., which may optionally comprise one or more suitable substituents, such as alkyl, alkenyl, alkynyl, alkoxy (for example methoxy, ethoxy etc.), halogen (for example fluorine, chlorine, bromine etc.), nitro and the like.

“Aralkyl” includes mono-, di-, triphenylalkyls such as benzyl, phenethyl, benzhydryl, trityl and the like, wherein the aromatic moiety may optionally comprise one or more suitable substituents, such as alkoxy (for example methoxy, ethoxy etc.), halogen (for example fluorine, chlorine, bromine etc.), nitro and the like.

“Alkyleneamine” includes linear or branched alkyleneamine groups which comprise up to 9 carbon atoms and may be represented by the formula

—(CₙH₂ₙ)—N—(CₘH₂ₘ)—

in which n and m may be identical or different and are an integer from 0 to 9, in which 1≦n+m≦9 applies, such as methyleneamine, ethyleneamine, dimethyleneamine, trimethyleneamine, methyleneethyleneamine, tetramethyleneamine, 1-methyltrimethyleneamine, 2-ethylethyleneamine, ethylenemethyleneamine, pentamethyleneamine, 2-methyltetramethyleneamine, isopropylethyleneamine, hexamethyleneamine and the like; preferred alkyleneamine residues have 2 carbon atoms, which are in terminal position. Dimethyleneamine is particularly preferred. The hydrogen atoms may also be replaced by substituents, such as for example halogen residues.

“Alkyleneimine” includes linear or branched alkyleneimine groups which comprise up to 9 carbon atoms and may be represented by the formula

—(CₙH₂ₙ₋₁)═N—(CₘH₂ₘ)— or the formula —(CₙH₂ₙ)—N═(CₘH₂ₘ₋₁)—

in which n and m may be identical or different and are an integer from 0 to 9, for which 1≦n+m≦9 applies, such as methyleneimine, ethyleneimine, dimethyleneimine, trimethyleneimine, methyleneethyleneimine, tetramethyleneimine, 1-methyltrimethyleneimine, 2-ethylethyleneimine, ethylenemethyleneimine, pentamethyleneimine, 2-methyltetramethyleneimine, isopropylethyleneimine, hexamethyleneimine and the like; preferred alkyleneimine residues have 2 carbon atoms, which are in terminal position. Dimethyleneimine is particularly preferred. The hydrogen atoms may also be replaced by substituents, such as for example halogen residues.

“Alkyleneamine” includes linear or branched alkyleneamine groups with up to 9 carbon atoms which may be represented by the formulae

—(CₙH₂ₙ₋₂)—N—(CₘH₂ₘ₋₂)—; —(CₒH₂₀)—N—(CₙH₂ₙ₋₂)—; —(CₙH₂ₙ₋₂)—N—(CₒH₂₀)—

in which n and m may be identical or different and are an integer from 2 to 9, for which m+n≦9 applies, and o is a number between 0 and 7 and o+n≦9 applies, such as for example vinyleneamine, methylenevinyleneamine, divinyleneamine, propenyleneamine (for example 1-propenyleneamine, 2-propenyleneamine), methylenepropenyleneamine, 1-methylpropenyleneamine, 2-methylpropenyleneamine, butenyleneamine, 2-ethylenepropenyleneamine, pentyleneamine, hexenyleneamine, vinylmethyleneamine and the like. The hydrogen atoms may also be replaced by substituents, such as for example halogen residues.

“Alkyleneimine” includes linear or branched alkyleneamine groups with up to 9 carbon atoms which may be represented by the formulae

—(CₙH₂ₙ₋₃)═N—(CₘH₂ₘ₋₂)—; —(CₒH₂ₒ₋₁)═N—(CₙH₂ₙ₋₂)—; —(CₙH₂ₙ₋₃)═N—(CₒH₂ₒ)—;

—(CₙH₂ₙ₋₂)—N═(CₘH₂ₘ₋₃)—; —(CₒH₂ₒ)—N═(CₙH₂ₙ₋₃)—; —(CₙH₂ₙ₋₂)—N═(CₒH₂ₒ₋₁)—

in which n and m are identical or different and are an integer from 2 to 9, for which m+n≦9 applies, and o is a number between 0 and 7 and o+n≦9 applies, such as for example vinylneimine, methylenevinyleneimine, propenyleneimine (for example 1-propenyleneimine, 2-propenyleneimine), methylenepropenyleneimine, 1-methylpropenyleneimine, 2-methylpropenyleneimine, butenyleneimine, 2-ethylenepropenyleneimine, pentyleneimine, hexenyleneimine, vinylmethyleneimine and the like. The hydrogen atoms may also be replaced by substituents, such as for example halogen residues.

“Hydroxyalkyleneamine” includes linear or branched alkylene residues, which have up to 9 carbon atoms, wherein at least one selected carbon atom is substituted with a hydroxy group; these residues may be reproduced by the formula

—(CₙH_{2n-z})(OH)_{z}—N—(CₘH_{2m-y})(OH)_{y}

in which n and m are identical or different and are an integer from 0 to 9, for which 1≦n+m≦9 applies, and z and y are identical or different and are an integer, for which 0≦z≦n and 0≦y≦m and y+z≧1 apply. Suitable examples of hydroxyalkyleneamine groups are hydroxymethyleneamine, hydroxyethyleneamine (for example 1 -hydroxyethyleneamine and 2-hydroxyethyleneamine), hydroxytrimethyleneamine (for example 1-hydroxytrimethylene [sic], 2-hydroxytrimethyleneamine and 3-hydroxytrimethyleneamine), hydroxytetramethyleneamine (for example 2-hydroxytetramethyleneamine), 2-hydroxy-2-methyltrimethyleneamine, hydroxypentamethyleneamine (for example 2-hydroxypentamethyleneamine), hydroxyhexamethyleneamine (for example 2-hydroxyhexamethyleneamine), methylenehydroxymethyleneamine, methylenehydroxyethyleneamine and the like. A particularly preferred compound is a lower hydroxyalkyleneamine with 2 carbon atoms and a nitrogen atom, wherein both carbon atoms are terminal. The hydrogen atoms may also be replaced by substituents, such as for example halogen residues.

“Hydroxyalkyleneimine” includes linear or branched alkylene residues, which have up to 9 carbon atoms, wherein at least one selected carbon atom is substituted with a hydroxy group; these residues may be reproduced by the formula

—(CₙH_{2n-z-1})(OH)_{z}═N—(CₘH_{2m-y})(OH)_{y}; —(CₙH_{2n-z-1})(OH)_{z}—N═(CₘH_{2m-y})(OH)_{y}

in which n and m are identical or different and are an integer from 0 to 9, for which 1≦n+m≦9 applies, and z and y are identical or different and are an integer, for which 0≦z≦n-1 and 0≦y≦m-1 and y+z≧1 apply. Suitable examples of such hydroxyalkyleneimine groups include hydroxymethyleneimine, hydroxyethyleneimine (for example 1-hydroxyethyleneimine and 2-hydroxyethyleneimine), hydroxytrimethyleneimine (for example 1 -hydroxytrimethylene, 2-hydroxytrimethyleneimine and 3-hydroxytrimethyleneimine), hydroxytetramethyleneimine (for example 2-hydroxytetramethyleneimine), 2-hydroxy-2-methyltrimethyleneimine, hydroxypentamethyleneimine (for example 2-hydroxypentamethyleneimine), hydroxyhexamethyleneimine (for example 2-hydroxyhexamethyleneimine), methylenehydroxymethyleneimine, methylenehydroxyethyleneimine and the like. A particularly preferred compound is a lower hydroxyalkyleneimine with 2 carbon atoms and a nitrogen atom, wherein both carbon atoms are terminal. The hydrogen atoms may also be replaced by substituents, such as for example halogen residues.

The residues X₃ and X₄ may preferably be selected such that esters are formed on the phosphono group or phosphino group. Suitable examples of such esters of the formulae (I), (IV) and (V) include suitable mono- and diesters, and preferred examples of such esters are alkyl esters (for example methyl ester, ethyl ester, propyl ester, isopropyl ester, butyl ester, isobutyl ester, hexyl ester etc.);

aralkyl esters (benzyl ester, phenethyl ester, benzhydryl ester, trityl ester etc.);

aryl esters (for example phenyl ester, tolyl ester, naphthyl ester etc.); aroylalkyl esters (for example phenacyl ester etc.); and silyl esters (for example of trialkylhalosilyl, dialkyldihalosilyl, alkyltrihalosilyl, dialkylarylhalosilyl, trialkoxyhalosilyl, dialkylaralkylhalosilyl, dialkoxydihalosilyl, trialkoxyhalosilyl etc.) and the like.

In the above esters, the alkane and/or arene moiety may optionally comprise at least one suitable substituent, such as halogen, alkoxy, hydroxy, nitro or the like.

X₃ and X₄ are preferably a metal of main groups I, II or III of the periodic system, ammonium, substituted ammonium or ammnonium compounds derived from ethylenediamine or amino acids. In other words, the salt compounds of the organophosphorus compounds are formed with organic or inorganic bases (for example sodium salt, potassium salt, calcium salt, aluminium salt, ammonium salt, magnesium salt, triethylamine salt, ethanolamine salt, dicyclohexylamine salt, ethylenediamine salt, N,N′-dibenzylethylenediamine salts etc.) as well as salts with amino acids (for example arginine salt, aspartic acid salt, glutarnic acid salt etc.) and the like.

The compounds according to the invention of the formulae (I), (IV) or (V) may be present in the protonated form thereof as an ammonium salt of organic or inorganic acids, such as hydrochloric acid, hydrobromic acid, sulfuric acid, nitric acid, methanesulfonic acid, p-toluenesulfonic acid, acetic acid, lactic acid, maleic acid, fumaric acid, oxalic acid, tartaric acid, benzoic acid etc.

The compounds according to the invention of the formulae (I), (IV) or (V) permit, for example for groups R₁, R₂, R₃, R₄, X₃, X₄ or A which contain double bonds or are chiral, the occurrence of steric isomers. The use according to the invention of the compounds includes all steric isomers, both as pure substances and in the form of mixtures.

The organophosphorus compounds are in particular suitable for the therapeutic and prophylactic treatment of infections in humans and animals caused by viruses, bacteria, uni- and multicellular parasites and fungi.

The compounds are active against unicellular parasites (protozoa), in particular against the causative organisms of malaria and sleeping sickness and of Chagas' disease, toxoplasmosis, amoebic dysentery, leishmaniases, trichomoniasis, pneumocystosis, balantidiasis, cryptosporidiosis, sarcocytosis, acanthamoebosis, naeglerosis, coccidiosis, giardiasis and lambliasis.

They are accordingly in particular suitable for the prophylactic treatment of malaria and of sleeping sickness and of Chagas' disease, of toxoplasmosis, amoebic dysentery, leishmaniases, trichomoniasis, pneumocystosis, balantidiasis, cryptosporidiosis, sarcocytosis, acanthamoebosis, naeglerosis, coccidiosis, giardiasis and lambliasis.

The active substances according to the invention may in particular be used against the following bacteria:

bacteria of the family Propionibacteriaceae, in particular of the genus Propionibacterium, in particular the species Propionibacterium acnes, bacteria of the family Actinomycetaceae, in particular of the genus Actinomyces, bacteria of the genus Cornynebacterium, in particular the species Corynebacterium diphtheriae and Corynebacterium pseudotuberculosis, bacteria of the family Mycobacteriaceae, of the genus Mycobacterium, in particular the species Mycobacterium leprae, Mycobacterium tuberculosis, Mycobacterium bovis and Mycobacterium avium, bacteria of the family Chlamydiaceae, in particular the species Chiamydia trachomatis and Chlamydia psittaci, bacteria of the genus Listeria, in particular the species Listeria monocytogenes, bacteria of the species Erysipelthrix rhusiopathiae, bacteria of the genus Clostridium, bacteria of the genus Yersinia, the species Yersinia pestis, Yersinia pseudotuberculosis, Yersinia enterocolitica and Yersinia ruckeri, bacteria of the family Mycoplasmataceae, of the genera Mycoplasma and Ureaplasma, in particular the species Mycoplasma pneumoniae, bacteria of the genus Brucella, bacteria of the genus Bordetella, bacteria of the family Neisseriaceae, in particular of the genera Neisseria and Moraxella, in particular the species Neisseria meningitides, Neisseria gonorrhoeae and Moraxella bovis, bacteria of the family Vibrionaceae, in particular of the genera Vibrio, Aeromonas, Plesiomonas and Photobacterium, in particular the species Vibrio cholerae, Vibrio anguillarum and Aeromonas salmonicidas, bacteria of the genus Campylobacter, in particular the species Campylobacter jejuni, Campylobacter coli and Campylobacter fetus, bacteria of the genus Helicobacter, in particular the species Helicobacter pylori, bacteria of the families Spirochaetaceae and Leptospiraceae, in particular of the genera Treponema, Borrelia and Leptospira, in particular Borrelia burgdorferi, bacteria of the genus Actinobacillus, bacteria of the family Legionellaceae, of the genus Legionella, bacteria of the family Rickettsiaceae and family Bartonellaceae, bacteria of the genera Nocardia and Rhodococcus, bacteria of the genus Dermatophilus, bacteria of the family Pseudomonadaceae, in particular of the genera Pseudomonas and Xanthomonas, bacteria of the family Enterobacteriaceae, in particular of the genera Escherichia, Klebsiella, Proteus, Providencia, Salmonella, Serratia and Shigella, bacteria of the family Pasteurellaceae, in particular of the genus Haemophilus, bacteria of the family Micrococcaceae, in particular of the genera Micrococcus and Staphylococcus, bacteria of the family Streptococcaceae, in particular of the genera Streptococcus and Enterococcus and bacteria of the family Bacillaceae, in particular of the genera Bacillus and Clostridium.

Organophosphorus compounds and the derivatives thereof are consequently suitable for treating diphtheria, acne vulgaris, listerioses, swine erysipelas in animals, gas gangrene in humans and animals, malignant oedema in humans and animals, tuberculosis in humans and animals, leprosy and further mycobacterioses in humans and animals, paratuberculosis in animals, plague, mesenterial lymphadenitis and pseudotuberculosis in humans and animals, cholera, legionnaires' disease, borreliosis in humans and animals, leptospiroses in humans and animals, syphilis, Campylobacter enteritis infections in humans and animals, Moraxella keratoconjunctivitis and serositis in animals, brucellosis of animals and humans, anthrax in humans and animals, actinomycosis in humans and animals, streptotrichoses, psittacosis/ortnithosis in animals, Q fever, ehrlichiosis.

Use is furthermore effective in the eradication of Helicobacter in ulcers of the gastrointestinal tract.

Combinations with another antibiotic may also be used to treat the above-stated diseases. Isoniazid, rifampicin, ethambutol, pyrazinarnide, streptomycin, protionamide and dapsone are in particular suitable for combination preparations with other antiinfective agents for the treatment of tuberculosis.

The active substances according to the invention are furthermore in particular usable in infections with the following viruses:

Parvoviridae: parvoviruses, dependoviruses, densoviruses, Adenoviridae: adenoviruses, mastadenoviruses, aviadenoviruses, Papovaviridae: papovaviruses, in particular papillomaviruses (“wart” viruses), polyomaviruses, in particular JC virus, BK virus and miopapovaviruses, Herpesviridae: all herpesviruses, in particular herpes simplex viruses, varicella-zoster viruses, human cytomegalovirus, Epstein-Barr viruses, all human herpesviruses, human herpesvirus 6, human herpesvirus 7, human herpesvirus 8, Poxiviridae: poxviruses, orthopoxviruses, parapoxviruses, molluscum contagiosum virus, aviviruses, capriviruses, leporipoxviruses, all primarily hepatotropic viruses, hepatitisviruses: hepatitis A viruses, hepatitis B viruses, hepatitis C viruses, hepatitis D viruses, hepatitis E viruses, hepatitis F viruses, hepatitis G viruses, hepadnaviruses: all hepatitisviruses, hepatitis B virus, hepatitis D viruses, Picornaviridae: picornaviruses, all enteroviruses, all polioviruses, all coxsackieviruses, all echoviruses, all rhinoviruses, hepatitis A virus, aphthoviruses, Calciviridae: hepatitis E viruses, Reoviridae: reoviruses, orbiviruses, rotaviruses, Togaviridae: togaviruses, alphaviruses, rubiviruses, pestiviruses, rubellavirus, Flaviviridae: flaviviruses, FSME virus, hepatitis C virus, Orthomyxoviridae: all influenza viruses, Paramyxoviridae: paramyxoviruses, morbillivirus, pneumovirus, measles virus, mumps virus, Rhabdoviridae: rhabdoviruses, rabies virus, lyssavirus, vascular stomatitisvirus, Coronaviridae: coronaviruses, Bunyaviridae: bunyaviruses, nairovirus, phlebovirus, uukuvirus, hantavirus, hantaan virus, Arenaviridae: arenaviruses, lymphocytic choriomeningitis virus, Retroviridae: retroviruses, all HTL viruses, human T-cell leukaemia virus, oncornaviruses, spumaviruses, lentiviruses, all HI viruses, Filoviridae: Marburg and Ebola virus, slow-virus infections, prions, oncoviruses and leukaemia viruses.

The organophosphorus compounds according to the invention are consequently suitable for combating the following viral infections:

eradication of papillomaviruses to prevent tumours, in particular tumours of the reproductive organs caused by papillomaviruses in humans, eradication of JC viruses and BK viruses, eradication of herpesviruses, eradication of human herpesvirus 8 to treat Kaposi's sarcoma, eradication of cytomegaloviruses before transplantations, eradication of Epstein-Barr viruses before transplantation and to prevent tumours associated with Epstein-Barr viruses, eradication of hepatitis viruses to treat chronic liver disease and to prevent liver tumours and cirrhosis of the liver, eradication of coxsackie-viruses in cardiomyopathy, eradication of coxsackie-viruses in diabetes mellitus patients, eradication of immunodeficiency viruses in humans and animals, treatment of accompanying infections in AIDS patients, treatment of respiratory tract inflammation of viral causation (laryngeal papilloma, hyperplasia, rhinitis, pharyngitis, bronchitis, pneumonia), of the sensory organs (keratoconjunctivitis), of the nervous system (poliomyelitis, meningoencephalitis, encephalitis, subacute sclerosing panencephalitis, SSPE, progressive multifocal leukoencephalopathy, lymphocytic choriomeningitis), of the gastrointestinal tract (stomatitis, gingivostomatitis, oesophagitis, gastritis, gastroenteritis, diarrhoea), of the liver and gall system (hepatitis, cholangitis, hepatocellular carcinoma), of the lymphatic tissue (mononucleosis, lymphadenitis), of the haemopoietic system, of the reproductive organs (mumps orchitis), of the skin (warts, dermatitis, herpes labialis, herpes febrilis, herpes zoster, shingles), of the mucous membranes (papillomas, conjunctival papillomas, hyperplasia, dysplasia), of the cardiovascular system (arteriitis, myocarditis, endocarditis, pericarditis), of the kidney/urinary system, of the reproductive organs (anogenital lesions, warts, genital warts, sharp condylomas, dysplasia, papillomas, cervical dysplasia, condyloma acuminatum, epidermodysplasia verruciformis), of the locomotory organs (myositis, myalgia), treatment of foot-and-mouth disease in cloven-hoofed animals, of Colorado tick fever, Dengue syndrome, of haemorrhagic fever, of early summer meningoencephalitis (FSME) and of yellow fever.

The described compounds, i.e. the organophosphorus compounds of the formulae (I), (IV) and (V) and esters and amides thereof on the phosphono or phosphino group and salts thereof exhibit strong cytotoxic activity against uni- and multicellular parasites, in particular against the causative organisms of malaria and sleeping sickness. The compounds according to the invention are accordingly usable for the treatment of infective diseases which are caused in humans and animals by viruses, bacteria, parasites and fungi. The compounds are also suitable for the prevention of diseases which are caused by viruses, bacteria, parasites and fungi, in particular for the prophylactic treatment of malaria and of sleeping sickness.

The organophosphorus compounds used according to the invention, which generally include for this purpose pharmaceutically acceptable salts, amides, esters, a salt of such an ester or also compounds which, on administration, provide the compounds used according to the invention as metabolites or breakdown products (also known as “prodrugs”), may be formulated for administration in any suitable manner analogous to known agents having an antiinfective action (mixed with a non-toxic, pharmaceutically acceptable excipient).

Pharmaceutically acceptable salts of the compounds include salts which the compounds of the formulae (I), (IV) and (V) according to the invention form in their protonated form as an ammonium salt of inorganic or organic acids, such as hydrochloric acid, sulfuric acid, citric acid, maleic acid, fumaric acid, tartaric acid, p-toluenesulfonic acid.

Particularly pharmaceutically suitable salts are also those formed by suitable selection of X₃ and X₄, such as sodium salt, potassium salt, calcium salt, ammonium salt, ethanolamine salt, triethylamine salt, dicyclohexylamine salt and salts of an amino acid such as arginine salt, aspartic acid salt, glutamic acid salt.

The activity of the substances is determined using a test system. This system is based upon in vitro measurement of the inhibition of growth of bacteria, parasites, viruses, fungi or plants. Test methods known to the person skilled in the art are in part used for this purpose.

For example, antimalarial activity is determined by measuring the inhibition of the growth of malaria parasites in blood cultures.

Antibacterial activity is determined on the basis of measuring the inhibition of bacterial growth on nutrient media and in liquid cultures.

Antiviral activity is determined on the basis of the formation of viral elements in cell cultures.

Fungicidal activity is determined on the basis of inhibition of fungal growth on nutrient media and in liquid cultures.

Some of the microorganisms which are to be investigated may only be investigated in animal models. In this case, we will then use the appropriate models.

Substances which exhibit activity in in vitro measurement systems are further investigated in in vivo models. Antiparasitic, antiviral, fungicidal or antibacterial activity is further evaluated in the appropriate animal models.

Screening for herbicidal activity is determined by means of algal systems and measurement of isoprene emissions from plants under standard conditions.

The pharmaceutically active agents may be prepared in dosage units in the form of pharmaceutical preparations. This means that the preparation is in the form of individual components, for example tablets, coated tablets, capsules, pills, suppositories and ampoules, the active substance content of which corresponds to a fraction or multiple of an individual dose. The dosage units may contain, for example 1, 2, 3 or 4 individual doses or 1/2, 1/3 or ¼ of an individual dose. An individual dose preferably contains the quantity of active substance which is administered at one time and usually corresponds to a whole, half, third or quarter of a daily dose.

Non-toxic, inert, pharmaceutically suitable excipients should be taken to mean solid, semi-solid or liquid diluents, fillers and formulation auxiliaries of all kinds.

Preferred pharmaceutical preparations which may be mentioned are tablets, coated tablets, capsules, pills, granules, suppositories, solutions, suspensions and emulsions, pastes, ointments, gels, creams, lotions, powders and sprays. Tablets, coated tablets, capsules, pills and granules may contain the active substances together with conventional excipients, such as (a) fillers and extenders, for example starches, lactose, cane sugar, glucose, mannitol and silica, (b) binders, for example carboxymethylcellulose, alginates, gelatine, polyvinylpyrrolidone, (c) humectants, for example glycerol, (d) suspending agents, for example agar-agar, calcium carbonate and sodium carbonate, (e) dissolution retardants, for example paraffin and (f) resorption accelerators, for example quaternary ammonium compounds, (g) wetting agents, for example cetyl alcohol, glycerol monostearate, (h) adsorbents, for example kaolin and bentonite and (i) lubricants, for example talcum, calcium and magnesium stearate and solid polyethylene glycols or mixtures of the substances stated in (a) to (i).

The tablets, coated tablets, capsules, pills and granules may be provided with conventional coatings and shells optionally containing opacifying agents and may also be composed such that they release the active substances only with a delay or preferably in a particular part of the intestinal tract, wherein polymeric substances and waxes may, for example, be used as the matrices.

The active substance or substances, optionally together with one or more of the above-stated excipients, may also be present in microencapsulated form.

In addition to the active substance or substances, suppositories may contain conventional water-soluble or water-insoluble excipients, for example polyethylene glycols, fats, for example cocoa butter and higher esters (for example C14 alcohol with C16 fatty acid) or mixtures of these substances.

In addition to the active substance or substances, ointments, pastes, creams and gels may contain conventional excipients, for example animal and vegetable fats, waxes, paraffins, starch, gum tragacanth, cellulose derivatives, polyethylene glycols, silicones, bentonites, silica, talcum and zinc oxide or mixtures of these substances.

In addition to the active substance or substances, powders and sprays may contain conventional excipients, for example lactose, talcum, silica, aluminium hydroxide, calcium silicate and polyamide powder or mixtures of these substances. Sprays may additionally contain conventional propellants, for example chlorofluorocarbons.

In addition to the active substance or substances, solutions and emulsions may contain conventional excipients, such as solvents, solubilising agents and emulsifiers, for example water, ethyl alcohol, isopropyl alcohol, ethyl carbonate, ethyl acetate, benzyl alcohol, benzyl benzoate, propylene glycol, 1,3-butylene glycol, dimethylformamide, oils, in particular cottonseed oil, peanut oil, corn oil, olive oil, castor oil and sesame oil, glycerol, glycerol formal, tetrahydrofurfuryl alcohol, polyethylene glycols and sorbitan fatty acid esters or mixtures of these substances.

For parenteral administration, the solutions and emulsions may also be present in sterile, isotonic form.

In addition to the active substance or substances, suspensions may contain conventional excipients, such as liquid diluents, for example water, ethyl alcohol, propylene glycol, suspending agents, for example ethoxylated isostearyl alcohols, polyoxyethylene sorbitol and sorbitan esters, microcrystalline cellulose, aluminium metahydroxide, bentonite, agar-agar and gum tragacanth or mixtures of these substances.

The stated formulations may also contain colorants, preservatives and odour- or flavour-enhanced additives, for example peppermint oil and eucalyptus oil, and sweeteners, for example saccharin.

The active substances of the formulae (I), (IV) and (V) should preferably be present in the pharmaceutical preparations listed above in a concentration of approx. 0.1 to 99.5 wt. %, preferably from approx. 0.5 to 95 wt. %, of the complete mixture.

Apart from the compounds of the formulae (I), (IV) and (V), the pharmaceutical preparations may also contain further pharmaceutical active substances.

The compounds may be used together with hitherto described substances having antibacterial, antiviral, antimycotic and antiparasitic properties. Such substances in particular include compounds which have already been used in therapeutic applications or are still used. Substances which are suitable for this purpose are in particular those listed in the Red List or in Simon/Stille, Antibiokia-Therapie in Klinik und Praxis, 9th edition, 1998, Schatauer Verlag, or on the Internet at http://www.customs.treas.gov/imp-exp/rulings/harmoniz/hrm129.html. The derivatives may in particular be present with penicillins, benzylpenicillin (penicillin G), phenoxypenicillins, isoxazolylpenicillins, aminopenicillins, ampicillin, amoxicillin, bacampicillin, carboxypenicillin, ticarcillin, temocillin, acylaminopenicillins, azlocillin, mezlocillin, piperacillin, apalcillin, mecillinam, cephalosporins, cefazolin group, cefuroxime group, cefoxitin group, cefoxitin, cefotetan, cefmetazole, latamoxef, flomoxef, cefotaxime group, cefozidime, ceftazidime group, ceftazidime, cefpirome, cefepime, conventional cephalosporins, cefsulodin, cefoperazone, oral cephalosporins of the cephalexin group, loracarbef, cefprozil, new broad-spectrum oral cephalosporins, cefixime, cefpodoxime-proxetil, cefuroxime-axetil, cefetamet, cefotiam-hexetil, cefdinir, ceftibuten, other β-lactam antibiotics, carbapenem, imipenem/cilastatin, meropenem, biapenem, aztreonam, β-lactamase inhibitors, clavulanic acid/amoxicillin, clavulanic acid/ticarcillin, sulbactam/ampicillin, tazobactam/piperacillin, tetracyclines, oxytetracycline, rolitetracycline, doxycycline, minocycline, chloramphenicol, aminoglycosides, gentamicin, tobramycin, netilmicin, amikacin, spectinomycin, macrolides, erythromycin, clarithromycin, roxithromycin, azithromycin, dirithromycin, spiramycin, josamycin, lincosamides, clindamycin, fusidic acid, glycopeptide antibiotics, vancomycin, teicoplanin, pristinamycin derivatives, fosfomycin, antimicrobial folic acid antagonists, sulfonamides, co-trimoxazole, trimethoprim, other diaminopyrimidine-sulfonamide combinations, nitrofurans, nitrofurantoin, nitrofurazone, gyrase inhibitors (quinolones), norfloxacin, ciprofloxacin, ofloxacin, sparfloxacin, enoxacin, fleroxacin, pefloxacin, lomefloxacin, Bay Y3118, nitroimidazoles, antimycobacterial agents, isoniazid, rifampicin, rifabutin, ethambutol, pyrazinamide, streptomycin, capreomycin, prothionamide, terizidone, dapsone, clofazimine, topical antibiotics, bacitracin, tyrothricin, polymyxins, neomycin, kanamycin, paromomycin, mupirocin, antiviral agents, acyclovir, ganciclovir, azidothymidine, didanosine, zalcitabine, thiacytidine, stavudine, ribavirin, idoxuridine, trifluridine, foscarnet, amantadine, interferons, tibol derivatives, proteinase inhibitors, antimycotics, polyenes, amphotericin B, nystatin, natamycin, azoles, azoles for septic therapy, miconazole, ketoconazole, itraconazole, fluconazole, UK-109,496, azoles for topical use, clotrimazole, econazole, isoconazole, oxiconazole, bifonazole, flucytosine, griseofilvin, ciclopirox olamine, tolnafnate, naftifine, terbinafine, amorolfine, anthraquinones, betulinic acid, semianthraquinones, xanthones, naphthoquinones, arylamino alcohols, quinine, quinidines, mefloquine, halofantrine, chloroquine, arnodiaquine, acridine, benzonaphthyridine, mepacrine, pyronaridine, dapsone, sulfonamides, sulfadoxine, sulfalenes, trimethoprin, proguanil, chlorproguanil, diaminopyrimidines, pyrimethamine, primaquine, aminoquinolines, WR 238,605, tetracycline, doxycycline, clindamycin, norfloxacin, ciprofloxacin, ofloxacin, artemisinin, dihydroartemisinin, 10b artemether, arteether, atresunate, atovaquone, suramin, melarsoprol, nifurtimox, stibogluconate sodium, pentamidine, amphotericin B, metronidazole, clioquinol, mebendazole, niclosamide, praziquantel, pyrantel, tiabendazole, diethylcarbamazine, ivermectin, bithionol, oxamniquine, metrifonate, piperazine, embonate.

The organophosphorus compounds may furthermore be present in the pharmaceutical preparations in combination with sulfonamide, sulfadoxine, artemisinin, atovaquone, quinine, chloroquine, hydroxychloroquine, mefloquine, halofantrine, pyrimethamine, armesin, tetracyclines, doxycycline, proguanil, metronidazole, praziquantel, niclosamide, mebendazole, pyrantel, tiabendazole, diethylcarbazine, piperazine, pyrivinium, metrifonate, oxamniquine, bithionol or suramin or two or more of these substances.

The above-stated pharmaceutical preparations are produced in the conventional manner using known methods, for example by mixing the active substance or substances with the excipient or excipients.

The stated preparations may be administered to humans and animals orally, rectally, parenterally (intravenously, intramuscularly, subcutaneously), intracisternally, intravaginally, intraperitoneally, topically (powders, ointments, drops) and for the treatment of infections in cavities, body cavities. Suitable preparations which may be considered are solutions for injections, solutions and suspensions for oral therapy, gels, infusion formulations, emulsions, ointments or drops. Topical treatment may be performed using ophthalmological and dermatological formulations, silver and other salts, ear drops, eye ointments, powders or solutions. Administration to animals may also be achieved via the feed or drinking water in suitable formulations. Gels, pulverulent formulations, powders, tablets, controlled-release tablets, premixes, concentrates, granules, pellets, tablets, boli, capsules, aerosols, sprays, inhalation formulations may also be used in humans and animals. The compounds according to the invention may also be incorporated into other supports, such as for example plastics (plastic chains for topical treatment), collagen or bone cement.

It has in general proved advantageous in both human and veterinary medicine to administer the active substances of the formula (I), (IV) and (V) in total quantities of approx. 0.05 to approx. 600, preferably of 0.5 to 200 mg/kg body weight per 24 hours, optionally in the form of two or more individual doses in order to achieve the desired results. An individual dose preferably contains the active substance or substances in quantities of approx. 1 to approx. 200, in particular of 1 to 60 mg/kg body weight. It may, however, be necessary to deviate from the stated dosages, in particular as a function of the nature and body weight of the patient to be treated, the nature and severity of the disease, the nature of the preparations and the route of administration of the pharmaceutical preparation and the period of time over which administration is performed.

In some cases, it may accordingly be sufficient to use less than the above-stated quantity of active substance, while in other cases more than the above-stated quantity of active substance must be used. The person skilled in the art will use his/her skill to determine the optimum dosage and route of administration required in each particular case.

The compounds according to the invention may be given to animals in conventional concentrations and preparations together with feed or feed preparations or with drinking water.

The compounds according to the invention are furthermore ideally usable as bactericides, fungicides and herbicides in plants.

It is in principle known to the person skilled in the art which synthetic pathway should be selected in order to produce the substances according to the invention.

The hydroxamic acids used below are either commercially available or were produced from the corresponding methyl esters using generally known methods (c.f. Houben-Weyl, Methoden der organischen Chemie, 4th edition, volume 8, pp. 686 et seq. and volume E5, pp. 1144 et seq.).

### EXAMPLE 1

### 2-(N-(N-Butanoyl-N-hydroxyaminoethyl)-N-methyl)-amino-1-hydroxyethyldimethylphosphine oxide (1)

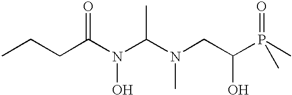

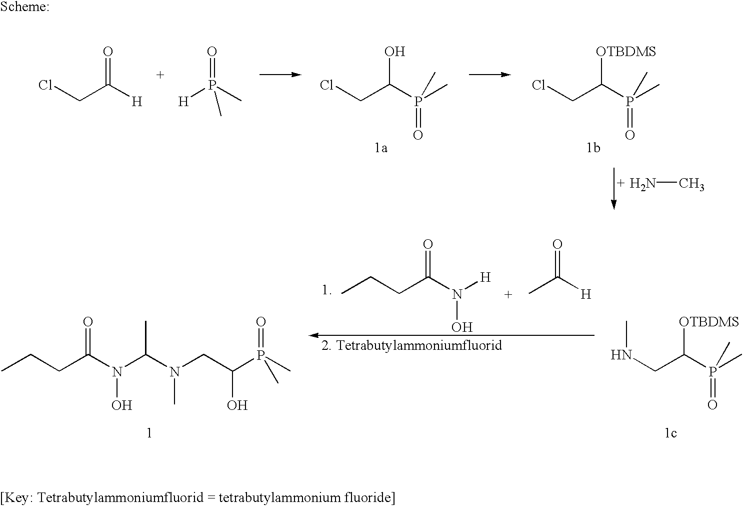

2-Chloro-1-hydroxyethyldimethylphosphine oxide 1a

7.81 g (0.1 mol) of dimethylphosphine oxide are added within 30 minutes with stirring and cooling to approx. 0.2 mol of an at least 45% aqueous chloroethanal solution. After a further 30 minutes' stirring at RT, water and unreacted chloroethanal are removed by vacuum distillation at an internal temperature of at most 50° C. The semi-solid residue is crystallised from acetone. 2-Chloro-1-hydroxyethyldimethylphosphine oxide (1a) is obtained in good yield as a colourless, waxy solid.

2-Chloro-1-(tert.-butyldimethylsiloxy)ethyldimethylphosphine oxide 1b

Reacting a solution of 13.00 g (83 mmol) of 1a in 100 ml of dry dichloromethane with 12.51 g (83 mmol) of tert.-butyldimethylsilyl chloride (TBDMSCl) in the presence of 6.56 g (83 mmol) of dry pyridine and a spatula-tipfull of 4-dimethylaminopyridine overnight at room temperature gives rise, after filtration and chromatographic treatment, to 2-chloro-1-(tert.-butyldimethylsiloxy)ethyldimethylphosphine oxide (1b) in good yield as a colourless, viscous oil.

2-Methylamino-1-(tert.-butyldimethylsiloxy)ethyldimethylphosphine oxide 1c

15.5 g (0.5 mol) of gaseous methylamine are condensed in a 250 ml glass autoclave into a solution of 14.59 g (55 mmol) of 1b in 60 ml of dry methanol. The sealed autoclave is then heated to 60° C. overnight. Once cool, the mixture is evaporated under a vacuum. The residue is digested repeatedly with ether. Evaporating the combined ether phases gives rise to 2-methylamino-1-(tert.-butyldimethylsiloxy)ethyldimethylphosphine oxide (1c) in moderate yield as a yellow oil which is further reacted without further purification.

2-(N-(N-Butanoyl-N-hydroxyaminoethyl)-N-methyl)-amino-1-hydroxyethyldimethylphosphine oxide (1)

A mixture of 1.55 g (15 mmol) of butanoylhydroxamic acid and 0.66 g (15 mmol) of freshly distilled acetaldehyde is combined at RT with 3.98 g (15 mmol) of 1c and shaken vigorously. Once the reaction has subsided, shaking is continued for a further 30 minutes. The resultant mixture is combined with a solution of 3.92 g (15 mmol) of tetrabutylammonium fluoride in 15 ml of dichloromethane and again shaken vigorously. After 10 minutes, the mixture is filtered, evaporated under a vacuum and all volatile constituents are removed from the residue at RT/0.1 torr. Crystallisation from dichloromethane/pentane gives rise to 2-(N-(N-butanoyl-N-hydroxyaminoethyl)-N-methyl)amino-1-hydroxyethyldimethylphosphine oxide (1) in poor yield as a yellowish, waxy solid.

### EXAMPLE 2

### N-(N-Benzoyl-N-hydroxyaminomethyl)aminomethyldimethylphosphine oxide (2)

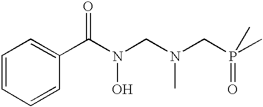

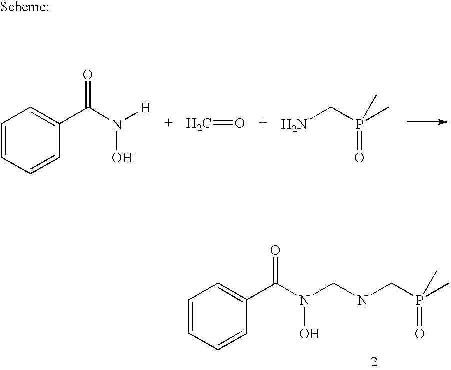

1.07 g (10 mmol) of aminomethyldimethylphosphine oxide (according to L. Maier, Phosphorus, Sulflir Silicon Relat. Elem. 53, 43-67 (1990)) are ground together with 1.37 g (10 mmol) of benzohydroxamic acid and 0.81 g (10 mmol) of 37% aqueous formaldehyde. The resultant oily mixture is dried in a desiccator over blue gel. Grinding the residue with a little dry methanol gives rise to a modest yield of almost colourless crystals of N-(N-benzoyl-N-hydroxyaminomethyl)aminomethyldimethylphosphine oxide (2).

### EXAMPLE 3

### N-((N-Cyclohexanoyl-N-hydroxy)aminomethyl)-N-methyl(aminomethylphosphinic acid, sodium salt (3)

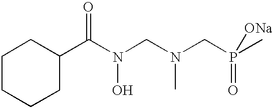

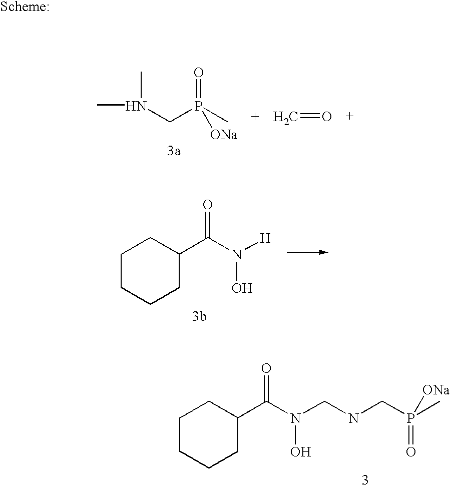

(N-Methylamino)methyl(methyl)phosphinic acid sodium salt 3a:

Compound 3a is obtained using a method described in the literature (R. Tyka, G. Haegele, J. Peters, Phosphorus Sulfur 1987, 34, 31-38).

(N-(N-Cyclohexanoyl-N-hydroxy)aminomethyl)-N-methyl(aminomethyl)methylphosphinic acid. sodium salt (3)

Compound 3 is produced using the above-stated general procedure for compound 2 from 1.74 g (12 mmol) of compound 3a, 1.72 g (12 mmol) of cyclohexanecarboxylic acid hydroxamate 3b and formalin. The product is obtained in slight yield.

### EXAMPLE 4

### N-[(4-N-Morpholinobutanoyl-N-hydroxy)aminomethyl]aminomethyl(methyl)phosphinic acid phenyl ester 4:

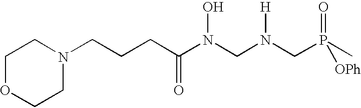

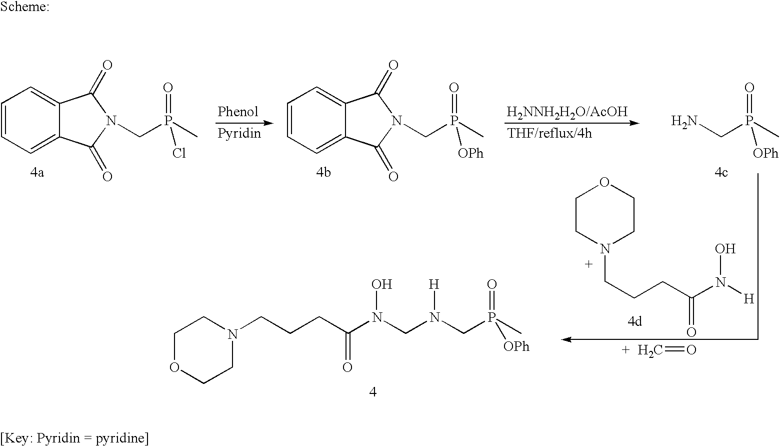

(N-Phthalimidoylmethyl)methylphosphinic acid chloride 4a:

Compound 4a was prepared using a method described in the literature (W. J. Moree, G. a. van der Marel, J. H. van Boom, R. H. J. Liskamp, Tetrahedron 49, 1993, 11055-11064 and literature cited therein) by means of a three-stage synthesis: 1. Arbuzov reaction of N-bromomethylphthalimide with diethylmethylphosphonite; 2. transesterification of the resultant protected (aminomethyl)methylphosphinic acid ethyl ester with trimethylbromosilane; 3. reaction of the silyl ester with oxalyl chloride to yield the desired (N-phthalimidoylmethyl)methylphosphinic acid chloride.

(N-Phthalimidoylmethyl)methylphosphinic acid phenyl ester 4b:

1.24 g (4.8 mmol) of 4a are dissolved under argon in thoroughly dried ether and cooled to 0° C. To this end, a common solution of 450 mg (4.8 mmol) of phenol and an equimolar quantity of dry pyridine are slowly added dropwise to the dry ether. After 1 hour at 0° C., the mixture is kept at RT for a further 4 hours. Subsequent aqueous treatment (extraction with 1 N HCl, 1 N NaOH, water and saturated common salt solution) gives rise to the desired product 4b in satisfactory yield in the form of a yellow oil which can be further used without purification.

(Aminomethyl)methylphosphinic acid phenyl ester 4c:

Synthesis proceeds in an analogous manner to that described in the literature (R. Hamilton, B. Walker, B. J. Walker, Bioorg. Med. Chem. Lett. 8 (1998) 1655-1660). To this end, the crude product 4b obtained from the previous procedure is dissolved in THF, combined with a large excess of hydrazine hydrate and then refluxed for 4 hours. The reaction mixture is then strongly evaporated and the residue redissolved with ethyl acetate. After treatment with an aqueous soda solution, the organic phase is separated and the aqueous phase extracted repeatedly with ethyl acetate. The combined organic phases are washed with water and dried with MgSO₄. The product 4c is obtained in moderate yield and is reacted without further purification.

3-(N-Morpholinyl)butyric acid hydroxatnate 4d:

Compound 4d is obtained according to Houben-Weyl, 4th edition, volume E5, pp. 1144 et seq. from 3-(N-morpholino)butyric acid methyl ester (prepared according to V. K. Tammara, M. M. Milind, A. M. Crider, A. Mansoor, J. Pharm. Sci 83, 644-648 (1994)) and hydroxylamine.

[(4-N-Morpholinobutanoyl-N-hydroxy)aminomethyl]aminomethl(methyl)phosphinic acid phenyl ester 4:

Phosphinic acid 4 is produced using the above-stated general procedure for compound 2 from 1.55 g (8.4 mmol) of compound 4c, 1.58 g (8.4 mmol) of hydroxamic acid 4d and formalin. The product is obtained in moderate yield.

### EXAMPLE 5

### 2-(N-2-Furanoyl-N-hydroxy-aminomethyl)amino-1-hydroxyethylmethylphosphinic acid, sodium salt 5

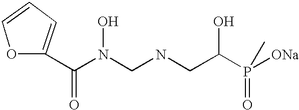

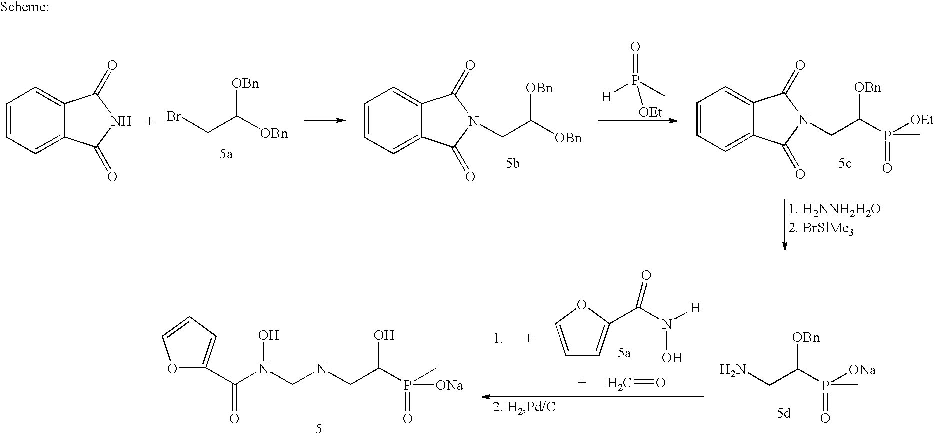

2-Bromoethanal dibenzyl acetal 5a:

49.2 (250 mmol) of 2-bromoethanal dimethyl acetal are dissolved in 54 g (500 mmol) of benzyl alcohol, 1 spatula-tipfull of p-toluenesulfonic acid is added and the mixture heated to 90° C. The resultant methanol is removed by distillation. Once the reaction is complete, ether is added and the mixture washed with dilute NaOH. The organic phase is separated and dried with sodium sulfate. Once the solvent has been removed, a crude product is obtained in good yield and of sufficient purity for further reaction.

2-(N-Phthalimidoyl)ethanal dibenzyl acetal 5b:

24.26 g (165 mmol) of phthalimide are slowly added to a solution of 9.5 g (170 mmol) of KOH in 300 ml of absolute ethanol. The mixture is then stirred for 1.5 hours, after which 49.32 g (195 mol) of 2-bromoethanal dibenzyl acetal 5a in 50 ml of absolute ethanol are added dropwise. The mixture is then heated to 60° C. for 2 hours. After cooling, water and ether are added and the organic phase separated. Said phase is dried with MgSO₄ and, once the solvent has evaporated, the residue is recrystallised from chloroform/ether. The product 5b is obtained as an almost white solid.

[2-(N-Phthalimidoyl)-1-benzyloxy]ethane(methyl)phosphinic acid ethyl ester 5c:

18.66 g (65 mmol) of 2-(N-phthalimidoyl)ethanal dibenzyl acetal 5b and 7.02 g (65 mmol) of methylphosphinic acid ethyl ester are dissolved in 150 ml of toluene and refluxed for 5 hours in a water separator. The solvent is removed under a vacuum and the residue chromatographed on silica gel. The product is then once more recrystallised from chloroform. The product is obtained as slightly yellowish crystals.

2-Amino-1-benzyloxyethane(methyl)phosphinic acid sodium salt 5d:

19.06 g (54 mmol) of compound 5c are reacted with an excess of hydrazine hydrate in analogous manner with the procedure described for compound 4c. The resultant crude product is dissolved under argon in dry methylene chloride and slowly combined at 0° C. with 2.5 equivalents of triethylbromosilane. After 1 hour at 0° C. and overnight at RT, the solvent and excess trimethylbromosilane are removed under a vacuum. The residue is redissolved in water, stirred for 1 h, combined with 1 equivalent of NaOH and the water stripped out under a vacuum. The residue is vacuum dried over phosphorus pentoxide in a desiccator. The desired product 5d is obtained in moderate yield.

Furan-2-carboxylic acid hydroxamate 5e:

Hydroxamic acid 5e is obtained according to Houben-Weyl, 4th edition, volume E5, pp. 1144 et seq. from furan-2-carboxylic acid methyl ester and hydroxylamine.

N-2-Furanoyl-N-hydroxy-aminomethyl)aminoethyl compound 5f: Compound 5f is produced using the above-stated general procedure for compound 2 from 3.46 g (18.6 mmol) of compound 5d, 2.38 g (18.6 mmol) of furan-2-carboxylic acid hydroxamate 5e and formalin. The product 5f is obtained in moderate yield.

2-[(N-2-Furanoyl-N-hydroxy)aminomethyl]amino-1-hydroxyethyl(methyl)phosphinic acid, sodium salt 5

In order to eliminate the benzyl group, 2.98 g (8.4 mmol) of the crude product 5f obtained from the preceding method are hydrogenated and worked up in analogous manner to the method for phosphinic acid 6b.

### EXAMPLE 6

### (2-(-N-(N-(3-Hydroxypropanoyl)-N-hydroxyaminoethyl)-N-methyl)amino-1-oxo)ethylmethylphosphinic acid, sodium salt (6)

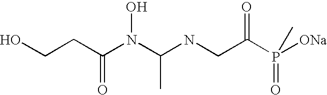

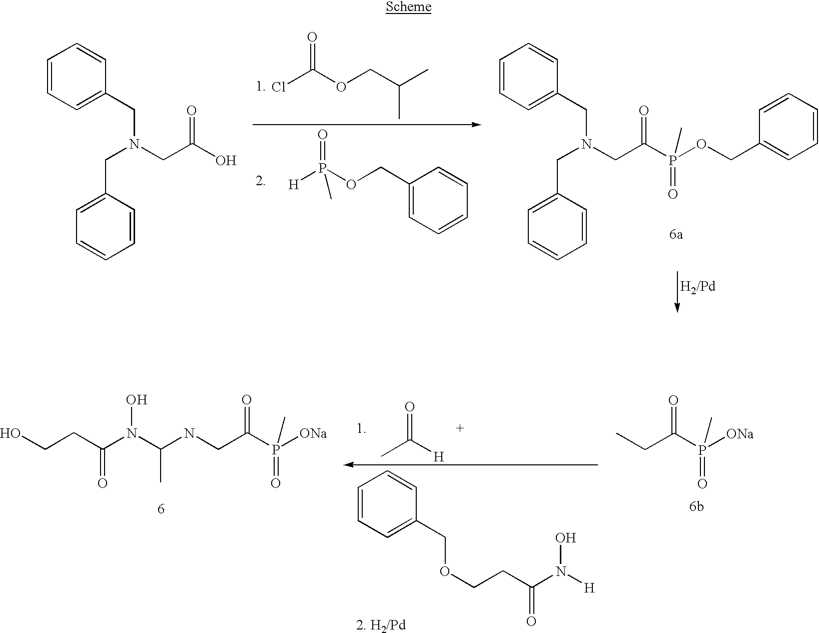

(2-N,N-Dibenzylamino-1-oxo)ethylmethylphosphinic acid benzyl ester (6a)

13.65 g (0.1 mol) of chloroformic acid isobutyl ester dissolved in 100 ml of dichloromethane are added dropwise within 30 minutes at 0° C. to a suspension of 25.5 g (0.1 mol) of N,N-dibenzylglycine and 10.1 g (0.1 mol) of triethylamine in 500 ml of dry dichloromethane. After 10 minutes' stirring at 0° C., the temperature is allowed to rise to RT and the suspension is combined with occasional cooling in succession with 10.1 g (0.1 mol) of triethylamine and 17.0 g (0.1 mol) of methylphosphinic acid benzyl ester. The mixture is stirred overnight at 20° C., then combined with 500 ml of ether and subsequently filtered. The filtrate is evaporated in a Rotavapor rotary evaporator. The oily residue is repeatedly digested with a little cold methanol, wherein the substance solidifies. After suction filtration and drying in a desiccator, (2-N,N-dibenzylamino-1-oxo)ethylmethylphosphinic acid benzyl ester (6a) is obtained in moderate yield as a yellowish solid.

(2-Amino-1-oxo)ethylmethylphosphinic acid, sodium salt (6b)

A solution of 17.60 g (45 mmol) of 6a in 400 ml of ethanol is subjected to hydrogenation at standard pressure at RT in the presence of 0.5 g of 20% palladium on activated carbon. Once hydrogen absorption has ceased, the catalyst is filtered out from the mixture. The filtrate is combined with 45 ml of 1 N NaOH and then evaporated under a vacuum in a Rotavapor rotary evaporator at a bath temperature of 50° C. The sodium salt of (2-amino-1-oxo)ethylmethylphosphinic acid (6b) is obtained in quantitative yield as a colourless solid.

(2-(N-(N-(3 -Hydroxypropanoyl)-N-hydroxyaminoethyl)-N-methyl)amino-1-oxo)ethylmethylphosphinic acid, sodium salt (6)

1.28 g (10 mmol) of 6b are ground with 1.95 g (10 mmol) of 3-benzyloxypropionohydroxamic acid (methyl ester: J. J. Bloomfield J. Org. Chem. 27, 2742-2746 (1962)) and 0.44 g (10 mmol) of freshly distilled acetaldehyde and, after gentle heating, dried in a desiccator over blue gel. After combining the resultant light yellow oil with a little absolute ethanol, crystals precipitate out in the freezing compartment and are washed with ethanol. Filtration gives rise to 6 in moderate yield as a virtually colourless solid.

2-(N-(N-Trifluoracetyl-N-hydroxyaminomethyl)-N-methyl)amino-1-oxoethyldimethylphosphine oxide (7)

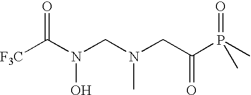

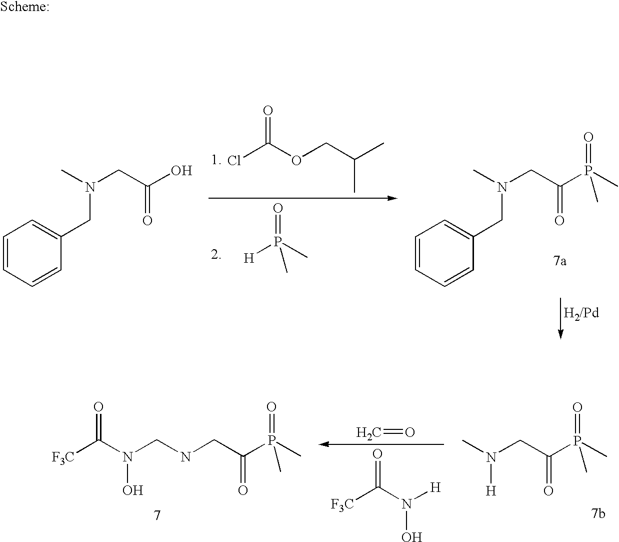

2-(N-Benzyl-N-methyl)amino-1-oxoethyldimethylphosphine oxide (7a)

In an analogous manner to 6a, 7a is obtained in moderate yield as a yellowish wax from 17.9 g (0.1 mol) of N-benzyl-N-methylglycine and 7.8 g (0.1 mol) of dimethylphosphine oxide.

2-Methylamino-1-oxoethyldimethylphosphine oxide (7b)

Standard pressure hydrogenation of 11.47 g (48 mmol) of 7a on 0.5 g of Pd/C (20%) in ethanol gives rise, after filtration and removal of the solvent, to 7b as a yellow oil in quantitative yield.

2-(N-(N-Trifluoracetyl-N-hydroxyaminomethyl)-N-methyl)amino-1-oxoethyldimethylphosphine oxide (7)

1.79 g (12 mmol) of (7b) are ground with 1.36 g (12 mmol) of trifluoroacetohydroxamic acid (according to J. J. Bloomfield J. Org. Chem. 27, 2742-2746 (1962)) and 12 mmol of 37% aqueous formaldehyde, after gentle heating, dried in a desiccator over blue gel. After combining the resultant light yellow oil with ethyl acetate, crystals form over the course of 3 weeks in the freezing compartment and are washed with a little ethyl acetate. Filtration gives rise to 7 in moderate yield as a colourless solid.

### EXAMPLE 8

### N-Methylphosphonic acid N-formyl-N-hydroxylaminoaldimine (9) or the sodium salt thereof

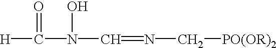

with R═H or Na⁺

N-Bromodiformamide (9d)

(9d) is known from the literature and may be prepared using the following synthesis:

N-Dibromomethylformamidium bromide (9a)

According to literature reference E. Allenstein, A. Schmidt, V. Beyl, Chem Ber. 1966, 99, 431-44, gaseous hydrogen bromide is introduced into liquid absolute hydrogen cyanide cooled to 0° C. with stirring until absorption ceases (the crystalline product gradually precipitates out). After leaving the mixture to stand for 12 hours at ambient temperature, it is carefully evaporated and the product (a) is adjusted to constant weight under an oil pump vacuum.

N-Formylformamidinium bromide (9b)

As described in the above literature reference, 3.7 g (0.044 mol) of absolute DMSO in 50 ml of dichloromethane are added dropwise to a suspension of 10.0 g (0.033 mol) of N-dibromomethylformamidinium bromide in 100 ml of absolute methylene chloride. After 15 hours' stirring a room temperature, the precipitate was filtered out, stirred in further methylene chloride, refiltered and washed with the same solvent. (9b) is adjusted to constant weight under an oil pump vacuum.

Diformamide (9c)

(c.f. E. Allenstein, V. Beyl, Chem. Ber. 1967, 100, 3551-63) A solution of 2.79 g of water in 350 ml of ether are added dropwise with stirring at room temperature to a suspension of 23.7 g (0.155 mol) of (9b) in 250 ml of absolute diethyl ether and stirred for 3 hours at the same temperature. Once the precipitate has been separated and washed with ether, the combined filtrates are dried over Na₂SO₄, evaporated under a vacuum at room temperature and combined with 50 ml of absolute petroleum ether. The colourless diformamide (c) is filtered out and rewashed with petroleum ether. (Yield: up to 70%, mp.: 39-40° C.).

N-Bromodiformamide (9d)

An ice-cooled suspension of 9.5 g (0.13 mol) of diformamide (9c) in 100 ml of absolute methylene chloride is combined with 26 g (0.163 mol) of bromine and 35.2 g (163 mol) of mercury(II) oxide and stirred for 1 hour at 0° C. The precipitate present is filtered again, washed with dichloromethane and the combined filtrates dried over sodium sulfate. After filtration from the desiccant, the mixture is evaporated at room temperature, cooled to −78° C., wherein yellow-orange coloured crystals precipitate, which are filtered out while cold. N-Bromodiformamide (9d) may be recrystallised from formic acid ethyl ester (yield approx. 7%).

N-Benzyldiformamide (9e)

0.018 mol of sodium are added with vigorous stirring to 30 ml of benzyl alcohol, stirring is continued until the remaining sodium has dissolved and 0.015 mol of N-bromodiformamide are then carefully added. After 2 hours' stirring at room temperature, the solution is evaporated under reduced pressure, the yellowish-orange oil is redissolved with toluene, the mixture washed with water and then re-evaporated. The crude product is further reacted without purification. (c.f. with regard to reaction conditions: C. Shin, K. Nanjo, T. Nishino, Y. Sato, J. Yoshimura, Bull. Chem. Soc. Jpn. 1975, 48, 2492-2495).

N-Methylphosphonic acid N-formyl-N-benzyloxyaminoaldimine (9f

Aminomethylphosphonic acid, dissolved in absolute methanol, is added dropwise to 1 equivalent of N-benzyldiformamide (9e), which has initially been introduced into the same solvent with a few crystals of p-toluenesulfonic acid. After 6 hours' stirring in a water bath, the mixture is neutralised with NaHCO₃ and evaporated under a vacuum. The resultant oil is chromatographed on cellulose in order to isolate (9f). N-Methylphosphonic acid N-formyl-N-(benzyloxy)aminoaldimine (9f) is obtained in slight yield.

N-Methylphosphonic N-formyl-N-hydroxylaminoaldimine (9g)

100 mg of N-methylphosphonic acid N-formyl-N-(benzyloxy)aminoaldimine (9f) dissolved in 30 ml of absolute ethanol are combined with 100 mg of 5% palladium on SrSO₄ and hydrogenated at standard pressure until no further hydrogen absorption is observed. Once the inorganic salts have been filtered out, the mixture is evaporated under a vacuum and an oil is obtained which can be recrystallised carefully from absolute acetone. (c.f. with regard to reaction conditions: D. E. Ames, T. F. Grey, J. Chem. Soc. 1955, 631-636).

Experiments demonstrate that the action of the compounds is based on the inhibition of the 1-deoxy-D-xylulose-5-phosphate (DOXP) metabolic pathway, which can be found in bacteria, parasites and fungi, but not in humans. The following Example consequently demonstrates the action of the compounds according to the invention on DOXP reductoisomerase

### EXAMPLE 9

The DOXP reductoisomerase of Helicobacter pylori was expressed as a recombinant protein in Helicobacter pylori. The activity of DOXP reductoisomerase was determined in a batch containing 100 mM tris-HCl (pH=7.5), 1 mM MnCl₂, 0.3 mM NADPH and 1 mM DOXP.

The oxidation of NADPH was here measured at 365 nm in a spectrophotometer. The inhibition studies were performed by measuring the activity of DOXP reductoisomerase in the presence of compounds 1 to 8 (see page 5) at various concentrations between 0. 1 and 100 μmol l⁻¹. The concentration at which the enzyme was semimaximally inhibited (IC₅₀) was calculated from the measurements. The results, i.e. the IC50 values, are shown in Table I.

TABLE I

| | IC50 (nM) of DOXP |
|---|---|
| | reductoisomerase from |
| Compound no. | Helicobacter pylori |
| 1 | 80 |
| 2 | 60 |
| 3 | 170 |
| 4 | 110 |
| 5 | 130 |
| 6 | 180 |
| 7 | 300 |
| 8 | 60 |

### EXAMPLE 10

The antimalarial activity of substances 1 to 8 (see page 5) was determined using in vitro cultures of the causative organism of malaria, Plasmodium falciparum. 200 μl of an asynchronous Plasmodium falciparum culture with a 0.4% blood parasite content and a heamatocrit of 2% were loaded into each of the wells of a 96 well microtitre plate. A serial dilution series of the compounds was then prepared in steps of three between concentrations of 100 and 0.14 μmol l⁻¹. The plates were incubated at 37° C., 3% CO₂ and 5% O₂ over a period of 48 hours. 30 μl of medium supplemented with 27 μCi ml⁻¹ of [³H]-hypoxanthine were then added to each well. After 24 hours' incubation, the parasites were harvested by filtration through glass fibre filters and the incorporated radioactivity was measured. Inhibition of parasite growth was measured as the percentage inhibition of tritium incorporation. Inhibition of parasite growth was expressed as the percentage inhibition of tritium incorporation relative to a comparison without the substance. The median inhibitory concentration (IC50) of the substance was determined by extrapolating the values. The results, i.e. the IC50 values, are listed in the following table II:

TABLE II

| | IC50 (nM) of malaria culture |
|---|---|
| Compound no. | (Plasmodium falciparum) |
| 1 | 580 |
| 2 | 420 |
| 3 | 4500 |
| 4 | 7300 |
| 5 | 6500 |
| 6 | 4400 |
| 7 | 1100 |
| 8 | 460 |

The herbicidal activity of the compounds according to the invention is also described below:

### EXAMPLE 11

Herbicidal activity is determined using standard methods.

Substances 1 to 8 listed on page 5 are again investigated.

Substance: 1

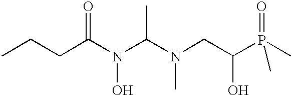

Water, pre-emergence

| gram/hectare | Rice | Lepidium | Echinochloa | Solanum | | |
|---|---|---|---|---|---|---|
| 1000 | 40% | 50% | 70% | 0% | | |
| Soil, pre-emergence | | | | | | |
| | | | Alopecurus | Avena | Cyperus | Setaria |
| gram/hectare | Zea mays | Beta vulgaris | myosuroides | fatua | esculentus | viridis |
| 500 | 0% | 30% | 40% | 0% | 0% | 0% |
| | Abutilon | Amaranthus | | Sinapis | Xanthium | |
| gram/hectare | theophrasti | retroflexus | Galium aperine | arvensis | strumarium | |
| 500 | 0% | 0% | 0% | 40% | 0% | |
| Soil, post-emergence | | | | | | |
| | | | Alopecurus | Avena | Cyperus | Setaria |
| gram/hectare | Zea mays | Beta vulgaris | myosuroides | fatua | esculentus | viridis |
| 500 | 80% | 50% | 60% | 50% | 0% | 95% |
| | Abutilon | Amaranthus | | Sinapis | Xanthium | |
| gram/hectare | theophrasti | retroflexus | Galium aperine | arvensis | strumarium | |
| 500 | 40% | 0% | 60% | 95% | 60% | |

Substance: 2

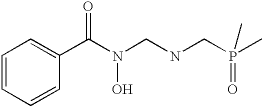

Water, pre-emergence

| gram/hectare | Rice | Lepidium | Echinochloa | Solanum | | |
|---|---|---|---|---|---|---|
| 1000 | 30% | 30% | 0% | 0% | | |
| Soil, pre-emergence | | | | | | |
| | | | Alopecurus | Avena | Cyperus | Setaria |
| gram/hectare | Zea mays | Beta vulgaris | myosuroides | fatua | esculentus | viridis |
| 500 | 0% | 50% | 0% | 0% | 0% | 0% |
| | Abutilon | Amaranthus | | Sinapis | Xanthium | |
| gram/hectare | theophrasti | retroflexus | Galium aperine | arvensis | strumarium | |
| 500 | 0% | 0% | 0% | 60% | 0% | |
| Soil, post-emergence | | | | | | |
| | | | Alopecurus | Avena | Cyperus | Setaria |
| gram/hectare | Zea mays | Beta vulgaris | myosuroides | fatua | esculentus | viridis |
| 500 | 70% | 50% | 60% | 70% | 0% | 90% |
| | Abutilon | Amaranthus | | Sinapis | Xanthium | |
| gram/hectare | theophrasti | retroflexus | Galium aperine | arvensis | strumarium | |
| 500 | 50% | 0% | 60% | 80% | 50% | |

Substance: 3

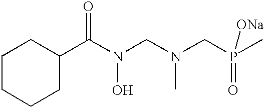

Water, pre-emergence

| gram/hectare | Rice | Lepidium | Echinochloa | Solanum | | |
|---|---|---|---|---|---|---|
| 4000 | 40% | 50% | 50% | 0% | | |
| Soil, pre-emergence | | | | | | |
| | | | Alopecurus | Avena | Cyperus | Setaria |
| gram/hectare | Zea mays | Beta vulgaris | myosuroides | fatua | esculentus | viridis |
| 2000 | 0% | 30% | 30% | 0% | 0% | 0% |
| | Abutilon | Amaranthus | | Sinapis | Xanthium | |
| gram/hectare | theophrasti | retroflexus | Galium aperine | arvensis | strumarium | |
| 2000 | 0% | 0% | 0% | 30% | 0% | |
| Soil, post-emergence | | | | | | |
| | | | Alopecurus | Avena | Cyperus | Setaria |
| gram/hectare | Zea mays | Beta vulgaris | myosuroides | fatua | esculentus | viridis |
| 2000 | 70% | 60% | 60% | 70% | 0% | 70% |
| | Abutilon | Amaranthus | | Sinapis | Xanthium | |
| gram/hectare | theophrasti | retroflexus | Galium aperine | arvensis | strumarium | |
| 2000 | 60% | 60% | 60% | 80% | 60% | |

Substance: 4

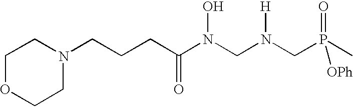

Water, pre-emergence

| gram/hectare | Rice | Lepidium | Echinochloa | Solanum | | |
|---|---|---|---|---|---|---|
| 4000 | 30% | 40% | 70% | 30% | | |
| Soil, pre-emergence | | | | | | |
| | | | Alopecurus | Avena | Cyperus | Setaria |
| gram/hectare | Zea mays | Beta vulgaris | myosuroides | fatua | esculentus | viridis |
| 2000 | 30% | 60% | 30% | 60% | 0% | 0% |
| | Abutilon | Amaranthus | | Sinapis | Xanthium | |
| gram/hectare | theophrasti | retroflexus | Galium aperine | arvensis | strumarium | |
| 2000 | 0% | 20% | 0% | 70% | 0% | |
| Soil, post-emergence | | | | | | |
| | | | Alopecurus | Avena | Cyperus | Setaria |
| gram/hectare | Zea mays | Beta vulgaris | myosuroides | fatua | esculentus | viridis |
| 2000 | 70% | 50% | 70% | 70% | 0% | 90% |
| | Abutilon | Amaranthus | | Sinapis | Xanthium | |
| gram/hectare | theophrasti | retroflexus | Galium aperine | arvensis | strumarium | |
| 2000 | 50% | 40% | 70% | 80% | 90% | |

Substance: 5

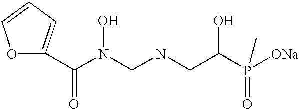

Water, pre-emergence

| gram/hectare | Rice | Lepidium | Echinochloa | Solanum | | |
|---|---|---|---|---|---|---|
| 4000 | 40% | 40% | 80% | 0% | | |
| Soil, pre-emergence | | | | | | |
| | Alopecurus | Avena | Cyperus | Setaria | | |
| gram/hectare | Zea mays | Beta vulgaris | myosuroides | fatua | esculentus | viridis |
| 2000 | 0% | 30% | 50% | 0% | 0% | 0% |
| | Abutilon | Amaranthus | | Sinapis | Xanthium | |
| gram/hectare | theophrasti | retroflexus | Galium aperine | arvensis | strumarium | |
| 2000 | 0% | 0% | 0% | 50% | 0% | |
| Soil, post-emergence | | | | | | |
| | | | Alopecurus | Avena | Cyperus | Setaria |
| gram/hectare | Zea mays | Beta vulgaris | myosuroides | fatua | esculentus | viridis |
| 2000 | 80% | 40% | 70% | 80% | 0% | 95% |
| | Abutilon | Amaranthus | | Sinapis | Xanthium | |
| gram/hectare | theophrasti | retroflexus | Galium aperine | arvensis | strumarium | |
| 2000 | 40% | 30% | 80% | 90% | 60% | |

Substance: 6

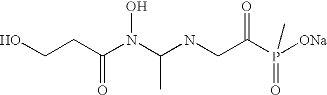

Water, pre-emergence

| gram/hectare | Rice | Lepidium | Echinochloa | Solanum | | |
|---|---|---|---|---|---|---|
| 4000 | 50% | 50% | 50% | 0% | | |
| Soil, pre-emergence | | | | | | |
| | | | Alopecurus | Avena | Cyperus | Setaria |
| gram/hectare | Zea mays | Beta vulgaris | myosuroides | fatua | esculentus | viridis |
| 2000 | 0% | 70% | 60% | 0% | 0% | 0% |
| | Abutilon | Amaranthus | | Sinapis | Xanthium | |
| gram/hectare | theophrasti | retroflexus | Galium aperine | arvensis | strumarium | |
| 2000 | 0% | 0% | 0% | 30% | 0% | |
| Soil, post-emergence | | | | | | |
| | | | Alopecurus | Avena | Cyperus | Setaria |
| gram/hectare | Zea mays | Beta vulgaris | myosuroides | fatua | esculentus | viridis |
| 2000 | 70% | 60% | 60% | 80% | 0% | 70% |
| | Abutilon | Amaranthus | | Sinapis | Xanthium | |
| gram/hectare | theophrasti | retroflexus | Galium aperine | arvensis | strumarium | |
| 2000 | 60% | 70% | 60% | 70% | 60% | |

Substance: 7

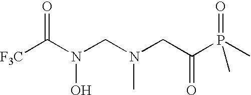

Water, pre-emergence

| gram/hectare | Rice | Lepidium | Echinochloa | Solanum | | |
|---|---|---|---|---|---|---|
| 4000 | 30% | 40% | 40% | 30% | | |
| Soil, pre-emergence | | | | | | |
| | | | Alopecurus | Avena | Cyperus | Setaria |
| gram/hectare | Zea mays | Beta vulgaris | myosuroides | fatua | esculentus | viridis |
| 2000 | 30% | 70% | 30% | 30% | 0% | 0% |
| | Abutilon | Amaranthus | | Sinapis | Xanthium | |
| gram/hectare | theophrasti | retroflexus | Galium aperine | arvensis | strumarium | |
| 2000 | 0% | 20% | 0% | 30% | 0% | |
| Soil, post-emergence | | | | | | |
| | | | Alopecurus | Avena | Cyperus | Setaria |
| gram/hectare | Zea mays | Beta vulgaris | myosuroides | fatua | esculentus | viridis |
| 2000 | 70% | 50% | 80% | 80% | 0% | 90% |
| | Abutilon | Amaranthus | | Sinapis | Xanthium | |
| gram/hectare | theophrasti | retroflexus | Galium aperine | arvensis | strumarium | |
| 2000 | 50% | 50% | 70% | 70% | 90% | |

Substance: 8

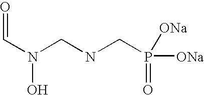

Water, pre-emergence

| gram/hectare | Rice | Lepidium | Echinochloa | Solanum | | |
|---|---|---|---|---|---|---|
| 2000 | 30% | 30% | 40% | 30% | | |
| Soil, pre-emergence | | | | | | |
| | | | Alopecurus | Avena | Cyperus | Setaria |
| gram/hectare | Zea mays | Beta vulgaris | myosuroides | fatua | esculentus | viridis |
| 1000 | 30% | 40% | 30% | 30% | 0% | 0% |
| | Abutilon | Amaranthus | | Sinapis | Xanthium | |
| gram/hectare | theophrasti | retroflexus | Galium aperine | arvensis | strumarium | |
| 1000 | 0% | 20% | 0% | 30% | 0% | |
| Soil, post-emergence | | | | | | |
| | | | Alopecurus | Avena | Cyperus | Setaria |
| gram/hectare | Zea mays | Beta vulgaris | myosuroides | fatua | esculentus | viridis |
| 1000 | 70% | 50% | 80% | 80% | 0% | 90% |
| | Abutilon | Amaranthus | | Sinapis | Xanthium | |
| gram/hectare | theophrasti | retroflexus | Galium aperine | arvensis | strumarium | |
| 1000 | 50% | 50% | 70% | 70% | 90% | |

1. Use of organophosphorus compounds of the general formula (I) 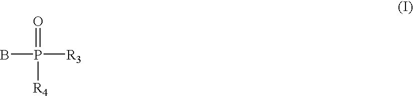
in which R₃ and R₄ are identical or different and are selected from the group which consists of hydrogen, substituted and unsubstituted alkyl with up to 26 carbon atoms, substituted and unsubstituted hydroxyalkyl with up to 26 carbon atoms, substituted and unsubstituted aryl, substituted and unsubstituted acyl, substituted and unsubstituted aralkyl, substituted and unsubstituted alkenyl with up to 26 carbon atoms, substituted and unsubstituted alkynyl with up to 26 carbon atoms, substituted and unsubstituted cycloalkyl, substituted and unsubstituted heterocyclic residue, halogen, OX₃ or OX₄, wherein X₃ or X₄ may be identical or different and are selected from the group which consists of hydrogen, substituted and unsubstituted alkyl with up to 26 carbon atoms, substituted and unsubstituted hydroxyalkyl with up to 26 carbon atoms, substituted and unsubstituted aryl, substituted and unsubstituted aralkyl, substituted and unsubstituted alkenyl with up to 26 carbon atoms, substituted and unsubstituted alkynyl with up to 26 carbon atoms, substituted and unsubstituted cycloalkyl, substituted and unsubstituted heterocyclic residue, a silyl, a cation of an organic and inorganic base, in particular a metal of main groups I, II or III of the periodic system, ammonium, substituted ammonium and ammonium compounds which are derived from ethylenediamine or amino acids, and B is selected from the group which consists of group (II) 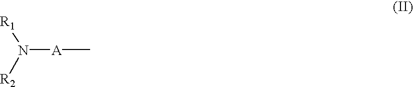
and group (III),
R₁—N═A— (III)
wherein A is selected from the group which consists of a substituted and unsubstituted alkyleneamine residue, a substituted and unsubstituted alkenyleneamine residue, a substituted and unsubstituted alkyleneimine residue and a substituted and unsubstituted alkenyleneimine residue and a hydroxyalkyleneimine residue, wherein the nitrogen atom is located in the chain which links the phosphorus atom with the nitrogen atom of the group 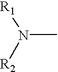
or the group R₁—N═, and in which R₁ and R₂ in group (II) are identical or different and R₁ and R₂ for group (II) and R₁ for group (III) are selected from the group which consists of hydrogen, substituted and unsubstituted alkyl, substituted and unsubstituted hydroxyalkyl, substituted and unsubstituted alkenyl, substituted and unsubstituted alkynyl, substituted and unsubstituted aryl, substituted and unsubstituted acyl, substituted and unsubstituted cycloalkyl, substituted and unsubstituted aralkyl, substituted and unsubstituted heterocyclic residue, halogen, OX₁ and OX₂, wherein X₁ and X₂ may be identical or different and are selected from the group which consists of hydrogen, substituted and unsubstituted alkyl, substituted and unsubstituted hydroxyalkyl, substituted and unsubstituted alkenyl, substituted and unsubstituted alkynyl, substituted and unsubstituted aryl, substituted and unsubstituted acyl, substituted and unsubstituted cycloalkyl, substituted and unsubstituted aralkyl, substituted and unsubstituted heterocyclic residue, and the pharmaceutically acceptable salts, esters and amides and salts of the esters for the treatment of infectious processes in humans and animals which are caused by viruses, bacteria, fungi or parasites and as a fungicide, bactericide or herbicide in plants.

2. Use according to claim 1, characterised in that the organophosphorus compounds are of the formula (IV) 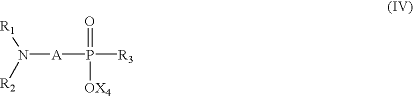
wherein R₁, R₂, R₃ and X₄ are defined as for formula (I), and A is selected from the group which consists of C—N—C, C═N—C, C—N═C, wherein the carbon atoms may be substituted with a hydroxy or alkyl group with up to 7 carbon atoms.

3. Use according to claim 2, characterised in that R₁ is a hydroxy group and R₂ is selected from the group which consists of acetyl and formyl.

4. Use according to claim 1, characterised in that the organophosphorus compounds are of the formula (V) 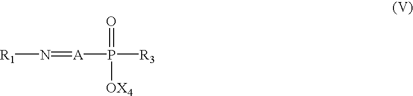
wherein R₁, R₃ and X₄ are defined as for formula (I), and A is selected from the group which consists of C—N—C, C═N—C, C—N═C, wherein the carbon atoms may be substituted with a hydroxy or alkyl group with up to 7 carbon atoms.

5. Use according to claim 4, characterised in that R₁ is selected from the group which consists of acetyl and formyl.

6. Use according to one of claims 1 to 5, characterised in that R₃ is selected from the group which consists of hydrogen, methyl, ethyl, hexadecanyl, octadecanyl, alkoxyaryl and OX₃, and X₃ is selected from the group which consists of hydrogen, sodium, potassium, methyl, ethyl, hexadecanyl, octadecanyl and alkoxyaryl, and, when both are present [sic], may be identical or different.

7. Use according to one of claims 1 to 6, characterised in that X₄ is selected from the group which consists of hydrogen, sodium, potassium, methyl, ethyl, hexadecanyl, octadecanyl and alkoxyaryl, and, when both are present [sic], may be identical or different.

8. Use according to claim 7, characterised in that the compound is selected from the group which consists of 2-(N-(N-butanoyl-N-hydroxyaminoethyl)-N-methyl)amino-1-hydroxyethyldimethylphosphine oxide, N-(N-benzoyl-N-hydroxyaminomethyl)-aminomethyldimethylphosphine oxide, N-((N-cyclohexanoyl-N-hydroxy)amino-methyl)-N-methyl(aminomethyl)methylphosphinic acid monosodium salt, N-[(4-N-morpholinobutanoyl-N-hydroxy)aminomethyl]aminomethyl(methyl)phosphinic acid phenyl ester, 2-(N-2-furanoyl-N-hydroxyaminomethyl)amino-1-hydroxyethylmethylphosphinic acid monosodium salt, (2-(N-(N-(3-hydroxypropanoyl)-N-hydroxyaminoethyl)-N-methyl)amino-1-oxo)ethylmethylphosphinic acid monosodium salt, 2-(N-(N-trifluoracetyl-N-hydroxyaminomethyl)-N-methyl)amino-1-oxoethyldimethylphosphine oxide, N-methylphosphonic acid N-formyl-N-hydroxylaminoaldimine and N-methylphosphonic acid N-formyl-N-hydroxylaminoaldimine sodium salt.

9. Use according to claims 1 to 8 for the treatment of infections caused by bacteria, viruses, fungi or uni- or multicellular parasites.

10. Use according to claim 9 for the treatment of infections which are caused by bacteria which are selected from the group which consists of bacteria of the family Propionibacteriaceae, in particular of the genus Propionibacterium, in particular the species Propionibacterium acnes, bacteria of the family Actinomycetaceae, in particular of the genus Actinomyces, bacteria of the genus Cornynebacterium, in particular the species Corynebacterium diphtheriae and Corynebacterium pseudotuberculosis, bacteria of the family Mycobacteriaceae, of the genus Mycobacterium, in particular the species Mycobacterium leprae, Mycobacterium tuberculosis, Mycobacterium bovis and Mycobacterium avium, bacteria of the family Chlamydiaceae, in particular the species Chlamydia trachomatis and Chlamydia psittaci, bacteria of the genus Listeria, in particular the species Listeria monocytogenes, bacteria of the species Erysipelthrix rhusiopathiae, bacteria of the genus Clostridium, bacteria of the genus Yersinia, the species Yersinia pestis, Yersinia pseudotuberculosis, Yersinia enterocolitica and Yersinia ruckeri, bacteria of the family Mycoplasmataceae, of the genera Mycoplasma and Ureaplasma, in particular the species Mycoplasma pneumoniae, bacteria of the genus Brucella, bacteria of the genus Bordetella, bacteria of the family Neisseriaceae, in particular of the genera Neisseria and Moraxella, in particular the species Neisseria meningitides, Neisseria gonorrhoeae and Moraxella bovis, bacteria of the family Vibrionaceae, in particular of the genera Vibrio, Aeromonas, Plesiomonas and Photobacterium, in particular the species Vibrio cholerae, Vibrio anguillarum and Aeromonas salmonicidas, bacteria of the genus Campylobacter, in particular the species Campylobacter jejuni, Campylobacter coli and Campylobacter fetus, bacteria of the genus Helicobacter, in particular the species Helicobacter pylori, bacteria of the families Spirochaetaceae and Leptospiraceae, in particular of the genera Treponema, Borrelia and Leptospira, in particular Borrelia burgdorferi, bacteria of the genus Actinobacillus, bacteria of the family Legionellaceae, of the genus Legionella, bacteria of the family Rickettsiaceae and family Bartonellaceae, bacteria of the genera Nocardia and Rhodococcus, bacteria of the genus Dermatophilus, bacteria of the family Pseudomonadaceae, in particular of the genera Pseudomonas and Xanthomonas, bacteria of the family Enterobacteriaceae, in particular of the genera Escherichia, Klebsiella, Proteus, Providencia, Salmonella, Serratia and Shigella, bacteria of the family Pasteurellaceae, in particular of the genus Haemophilus, bacteria of the family Micrococcaceae, in particular of the genera Micrococcus and Staphylococcus, bacteria of the family Streptococcaceae, in particular of the genera Streptococcus and Enterococcus and bacteria of the family Bacillaceae, in particular of the genera Bacillus and Clostridium, and in the eradication of Helicobacter in ulcers of the gastrointestinal tract.

11. Use according to claim 9 for the treatment of infections which are caused by viruses which are selected from the group which consists of viruses of the genus Parvoviridae, in particular parvoviruses, dependoviruses, densoviruses, viruses of the genus Adenoviridae, in particular adenoviruses, mastadenoviruses, aviadenoviruses, viruses of the genus Papovaviridae, in particular papovaviruses, in particular papillomaviruses (“wart” viruses), polyomaviruses, in particular JC virus, BK virus and miopapovaviruses, viruses of the genus Herpesviridae in particular herpes simplex viruses, varicella-zoster viruses, human cytomegalovirus, Epstein-Barr viruses, human herpesvirus 6, human herpesvirus 7, human herpesvirus 8, viruses of the genus Poxiviridae, in particular poxviruses, orthopoxviruses, parapoxviruses, molluscumn contagiosum virus, aviviruses, capriviruses, leporipoxviruses, primarily hepatotropic viruses, in particular hepatitisviruses, such as hepatitis A viruses, hepatitis B viruses, hepatitis C viruses, hepatitis D viruses, hepatitis E viruses, hepatitis F viruses, hepatitis G viruses, hepadnaviruses, in particular all hepatitisviruses, such as hepatitis B virus, hepatitis D viruses, viruses of the genus Picomaviridae, in particular picornaviruses, all enteroviruses, all polioviruses, all coxsackie-viruses, all echoviruses, all rhinoviruses, hepatitis A virus, aphthoviruses, viruses of the genus Calciviridae, in particular hepatitis E viruses, viruses of the genus Reoviridae, in particular reoviruses, orbiviruses, rotaviruses, viruses of the genus Togaviridae, in particular togaviruses, alphaviruses, rubiviruses, pestiviruses, rubellavirus, viruses of the genus Flaviviridae, in particular flaviviruses, FSME virus, hepatitis C virus, viruses of the genus Orthomyxoviridae, in particular all influenza viruses, viruses of the genus Paramyxoviridae, in particular paramyxoviruses, morbillivirus, pneumovirus, measles virus, mumps virus, viruses of the genus Rhabdoviridae, in particular rhabdoviruses, rabies virus, lyssavirus, vascular stomatitisvirus, viruses of the genus Coronaviridae, in particular coronaviruses, viruses of the genus Bunyaviridae, in particular bunyaviruses, nairovirus, phlebovirus, uukuvirus, hantavirus, hantaan virus, viruses of the genus Arenaviridae, in particular arenaviruses, lymphocytic choriomeningitis virus, viruses of the genus Retroviridae, in particular retroviruses, all HTL viruses, human T-cell leukaemia virus, oncornaviruses, spumaviruses, lentiviruses, all HI viruses, viruses of the genus Filoviridae, in particular Marburg and Ebola virus, slow-viruses, prions, oncoviruses and leukaemia viruses.

12. Use according to claim 9 for the prevention and treatment of infections caused by unicellular parasites, namely the causative organisms of malaria, sleeping sickness, Chagas' disease, toxoplasmosis, amoebic dysentery, leishmaniases, trichomoniasis, pneumocystosis, balantidiasis, cryptosporidiosis, sarcocytosis, acanthamoebosis, naeglerosis, coccidiosis, giardiasis and lambliasis.

13. A process for the treatment of infectious diseases caused by bacteria, fungi or parasites in which a therapeutically effective quantity of a compound according to one of claims 1 to 8 is administered to a patient suffering from an infection caused by bacteria, viruses, fungi or parasites.
